SEQUENCE LISTING (16 sequences)

SEQ ID NO: 1 - DNA, 501 bp, Clostridium thermocellum
gcaaatacaccggtatcaggcaatttgaaggttgaattctacaacagcaatccttcagatactactaactcaatcaatcctcagttcaaggttactaataccggaagcagtgcaattgatttgtccaaactcacattgagatattattatacagtagacggacagaaagatcagaccttctggtgtgaccatgctgcaataatcggcagtaacggcagctacaacggaattacttcaaatgtaaaaggaacatttgtaaaaatgagttcctcaacaaataacgcagacacctaccttgaaataagctttacaggcggaactcttgaaccgggtgcacatgttcagatacaaggtagatttgcaaagaatgactggagtaactatacacagtcaaatgactactcattcaagtctgcttcacagtttgttgaatgggatcaggtaacagcatacttgaacggtgttcttgtatggggtaaagaacccggtggcagtgtagta

SEQ ID NO: 2 - DNA, 114 bp, Clostridium thermocellum
aatgcaacaccgaccaagggagcaacaccaacaaatacagctacgccgacaaaatcagctacggctacgcccaccaggccatcggtaccgacaaacacaccgacaaacacaccg

SEQ ID NO: 3 - DNA, 102 bp, Clostridium thermocellum
ccatcaacacagcctgtaacaacaccacctgcaacaacaaaaccacctgcaacaacaaaaccacctgcaacaacaataccgccgtcagatgatccgaatgca

SEQ ID NO: 4 - protein, 167 aa, Clostridium thermocellum
ANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVV

SEQ ID NO: 5 - protein, 38 aa, Clostridium thermocellum
NATPTKGATPTNTATPTKSATATPTRPSVPTNTPTNTP

SEQ ID NO: 6 - protein, 34 aa, Clostridium thermocellum
PSTQPVTTPPATTKPPATTKPPATTIPPSDDPNA

SEQ ID NO: 7 - protein, 12 aa, Human immunodeficiency virus type 1 - gp41 epitope
LGIWGCSGKLIC

SEQ ID NO: 8 - DNA, 37 bp, Artificial Sequence
  pcr primer I for constructing a library of chimeric proteins (Example 3)
gagatatatcatgagcgctaatacaccggtatcaggc

SEQ ID NO: 9 - DNA, 63 bp, Artificial Sequence
  pcr primer II for constructing a library of chimeric proteins (Example 3)
tagcagggatccttavnnvnnvnnvnnvnnvnnvnnvnnactgcctacaggctgtgttgatgg

SEQ ID NO: 10 - protein, 8 aa, Artificial Sequence
  The CS1 epitope within a4b1 integrin
EILDVPST

SEQ ID NO: 11 - DNA, 29 bp, Artificial Sequence
  pcr primer III for constructing a libarary of chemiric peptides (see Example 14)
ccgcaccatggcaaatacaccggtattca

SEQ ID NO: 12 - DNA, 31 bp, Artificial Sequence
  pcr primer IV for constructing a libarary of chemiric peptides (see Example 14)
tcgcggatccttatactacactgccaccggg

SEQ ID NO: 13 - DNA, 42 bp, Artificial Sequence
  pcr primer V for constructing a libarary of chemiric peptides (see Example 14)
gcccgttgaaaccgtgccggtcacttttacaaatgttccttt

SEQ ID NO: 14 - DNA, 41 bp, Artificial Sequence
  pcr primer VI for constructing a libarary of chemiric peptides (see Example 14)
gtgaccggcacggtttcaacgggcgacacctaccttgaaat

SEQ ID NO: 15 - protein, 8 aa, Artificial Sequence
  mutation in loop 5/6 of the protein comprising SEQ ID NO 1
VTGTVSTG

SEQ ID NO: 16 - protein, 8 aa, Clostridium thermocellum
MSSSTNNA

(19) United States
(12) Patent Application Publication
(10) Pub. No.: US 2005/0118729 A1
(43) Pub. Date: Jun. 2, 2005

(54) Microarrays of cellulose binding chimeric proteins and methods of use thereof

(76) Inventor: Ely Morag, Misgav Dov (IL)

Correspondence Address: WINSTON & STRAWN;PATENT DEPARTMENT, 1400 L STREET, N.W., WASHINGTON, DC, 20005-3502, US

(21) Appl. No.: 10/933,404

(22) Filed: Sep. 3, 2004

Related U.S. Application Data
(63) Continuation of application No. PCT/IL03/00177, filed on Mar. 6, 2003.
(60) Provisional application No. 60/362,061, filed on Mar. 7, 2002.

(51) Int. Cl. G01N 33/543

(52) U.S. Cl. 436/518

(57) ABSTRACT

The present invention provides microarrays of proteins comprising a cellulose binding region as a means for binding to a cellulase substrate such as cellulose. The cellulose binding region may further contain biologically active exogenous moities introduced therein while maintaining the ability to bind cellulase substrates with high affinity. In addition, methods of construction of the microarrays and using the microarrays are disclosed. The microarrays of the invention are particularly useful for displaying peptide libraries, random or rationally designed, and for high throughput screening for ligands, epitopes or ligand binding sites.

### CROSS REFERENCE TO RELATED APPLICATIONS

This application is a continuation of International application no. PCT/IL03/00177 filed Mar. 6, 2003, and claims the benefit of provisional application No. 60/362,061 filed Mar. 7, 2002. The entire content of each application is expressly incorporated herein by reference thereto.

### FIELD OF THE INVENTION

The present invention relates to microarrays of proteins comprising a cellulose-binding region as a means for binding to a cellulase substrate such as cellulose. The invention further relates to methods of construction of the microarrays and to the use of the microarrays for displaying peptide libraries and for high throughput screening for ligands, epitopes or ligand binding sites.

### BACKGROUND OF THE INVENTION

Microarray technology is currently a preferred method for the efficient study and characterization of a large repertoire of genes and proteins. Microarrays consist of an ordered arrangement of known biological active elements, immobilized on a substrate. The technology of microarrays is particularly suitable for detection, screening, sequence analysis and generation of libraries.

Arrays of Biological Molecules

DNA microarray technology, particularly for nucleic acid hybridization assays (‘DNA-Chips’), is well known in the art. A method for synthesizing oligonucleotides on a solid phase in order to generate an array of diverse oligonucleotides is disclosed in U.S. Pat. No. 5,424,186. The method involves providing oligonucleotide molecules, wherein each molecule is coupled to a photoremovable protecting group and is immobilized on a solid substrate, and irradiating predefined region(s) of the substrate to remove the protecting group at that region(s).

A method of preparing an oligonucleotide bound to a solid support is disclosed in U.S. Pat. No. 5,436,327. The method involves attachment a 2′-deoxynucleoside reagent to a support which carries hydroxyl groups by a covalent phosphodiester link and synthesizing on the supported nucleoside an oligonucleotide chain including protecting groups.

A spatially addressable array of immobilized compounds, preferably polynucleotides, and a tracer is disclosed in U.S. Pat. No. 6,245,518. The structures of the compounds are identifiable by their spatial addresses and the amount of the tracer moiety attached at each address is proportional to the amount of compound attached at that address.

A method of fabricating an addressable array of biopolymers on a substrate using a biomonomer with a first linking group is disclosed in U.S. Pat. No. 6,306,599. The first linking group must be activated for linking to a substrate bound moiety, so as to form the addressable array.

U.S. Pat. No. 6,475,809 discloses a method for screening a plurality of proteins for interactions with a fluid sample comprising the step of providing an array of selected proteins displayed over a surface within protein immobilization regions and border regions surrounding and separating each protein immobilization region from one another and effectively resistant to non-specific protein binding. The method further comprises the steps of exposing the array of proteins to the fluid sample and detecting interaction(s) within the array.

Proteins Comprising a Cellulose Binding Region

The cellulosome is a cellulase system in cellulolytic bacteria that contains discrete multifunctional multienzyme complexes, whereas each complex is an assembly of subunits devoted to the efficient degradation of cellulose. Some of these units have distinct cellulose binding regions. Over 120 different cellulose binding region sequences have been identified and grouped into at least 10 families on the basis of sequence similarities. By means of the binding affinity between the cellulose-binding region and the cellulose, the cellulase can be immobilized on a matrix coated with cellulose.

Chimeric cellulose binding proteins and their applications for protein isolation are known in the art. For example, a fusion protein comprising a polypeptide and an amino acid sequence having a substrate binding region of a cellulase with the proviso that the substrate binding region is essentially lacking in cellulase activity is disclosed in U.S. Pat. No. 5,202,247. The preferred cellulose binding region is derived from Cellulomonas Fimi. Methods for immobilization, purification and other applications involving use of the fusion protein are disclosed in U.S. Pat. Nos. 5,137,819; 5,340,731; 5,928,917 among others.

A polynucleotide encoding a cellulose binding domain (CBD) derived from Clostridium Cellulovorans is disclosed in U.S. Pat. No. 5,496,934. Proteins and fusion proteins comprising a CBD, methods of purification these proteins or complexes containing same, kits and methods of detection based on said proteins among other applications utilizing said proteins, are disclosed for example in U.S. Pat. Nos. 5,670,623; 5,719,044; 5,738,984 and 5,837,814.

U.S. Pat. No. 6,407,208 discloses a chimeric protein comprising a cellulose-binding domain generated from cellulobiohydrolase-I of Trichoderma konigii G39 which has a C-terminal site joined to a peptide containing arginine-glutamate-aspartate and an N-terminal joined to a thioredoxin.

International Patent Application No. WO96/13524 discloses a modified cellulose binding domain (CBD), particularly a biotinylated CBD having a binding affinity to cellulose similar to the binding affinity of an unmodified CBD. The invention relates to a process for over expression of the soluble form of CBD of the scaffoldin subunit from the cellulosome of Clostridium thermocellum in suitable host cells.

Nowhere in the background art is it taught or suggested that a protein comprising a cellulose-binding region may be used to attach molecules in a microarray as a means for construction of molecular libraries, such as DNA and peptide libraries, and for high throughput methods of screening for ligands or epitopes or ligand binding sites.

### SUMMARY OF THE INVENTION

According to some aspects of the present invention microarrays are provided comprising a plurality of proteins comprising a cellulose binding region.

According to other aspects of the present invention methods are provided for screening of these microarrays and identifying ligands, epitopes and ligand binding sites.

Particularly, the present invention provides addressable arrays comprising a plurality of chimeric proteins. More particularly the present invention provides addressable arrays of a plurality of polypeptides comprising a cellulose binding region wherein the polypeptides further comprise at least one additional exogenous biologically active moiety while maintaining the capacity of binding cellulase substrates.

A major advantage of the microarrays of the present invention over the existing technologies is simplicity of fabrication, as in most embodiments the microarrays of the present invention consist essentially of cellulose polymers and a plurality of proteins comprising a cellulose binding region. Furthermore, fabrication of the microarrays of the present invention does not require any chemical modification or purification of the proteins prior to their immobilization onto the solid support.

An additional advantage of the microarrays of the present invention is the low cost of cellulose polymers. Hence, the microarrays of the present invention provide an ideal platform for applications which require a large workload at high speed, high sensitivity and low cost.

Yet another advantage of the microarrays of the present invention over the existing technologies is that the biological moieties are exposed to the external environment in an accessible orientation while immobilized.

The microarrays of the present invention are particularly efficient and stable under harsh and extreme conditions, such as high temperature, high urea concentrations, high SDS and TWEEN® concentrations.

The microarrays and the methods of the present invention are suitable for use with many of the cellulose binding regions, cellulose matrices and fabrication technologies known in the art. Preferably, the cellulose binding region used in the compositions and methods of the present invention are derived from a complex devoid of cellulase activity. The microarrays of the present invention comprising cellulose binding regions derived from Clostridium thermocellum have proven particularly effective and have yielded unexpectedly advantageous exemplary results.

According to one embodiment, the present invention provides a microarray comprising a plurality of chimeric polypeptides comprising a cellulose binding region, capable of attaching to a cellulose polymer with high affinity.

According to another embodiment, the microarrays of the present invention comprise a plurality of chimeric polypeptides comprising a cellulose binding region attached to a solid phase support comprising a cellulose polymer.

According to yet another embodiment, the microarrays of the present invention comprise cellulose polymers selected from the group consisting of: cellulose homopolymers, cellulose heteropolymers, cellulose acetate, microcrystalline cellulose, lignin, starch, xylane.

According to another embodiment, the present invention provides microarrays consisting essentially of a plurality of chimeric polypeptides comprising a cellulose binding region and a cellulose coated solid support.

According to another embodiment, the microarrays of the present invention comprise a plurality of chimeric polypeptides comprising a cellulose binding region wherein the cellulose binding region further contains at least one exogenous peptide sequence introduced therein while maintaining the capacity to bind cellulase substrates with high affinity. Preferably, the peptide comprises a sequence of 5 to 50 amino acid residues, more preferably a sequence of about 15 to 30 amino acid residues and most preferably a sequence of about 8 to 12 amino acid residues. Particularly, each polypeptide is attached to a solid phase support comprising a cellulase substrate wherein the exogenous peptide introduced therein is exposed to the external environment.

According to yet another embodiment, the present invention provides microarrays comprising a plurality of chimeric proteins wherein each chimeric protein comprises a cellulose binding region and at least one additional exogenous biologically active moiety fused to the cellulose binding region while maintaining the capacity to bind cellulose polymers with high affinity.

According to yet another embodiment, the microarrays of the present invention comprise a plurality of chimeric proteins comprising a cellulose binding region wherein at least one additional biologically active moiety is covalently linked to the cellulose binding region either directly or via a linker.

In some embodiments, the chimeric proteins comprise at least two biologically active moieties.

In some embodiments, the at least two biologically active moieties in each protein may be the same and in other embodiments the at least two biologically active moieties in each protein may be different from each other.

The biologically active moieties in each chimeric protein are exposed to the external environment in an orientation accessible to the external medium, while the protein is immobilized onto a polymer comprising a substrate binding a cellulose binding region.

According to yet another embodiment, the microarrays of the present invention are spatially addressable.

According to a preferred embodiment, the biologically active moiety is selected from the group consisting of enzymes, proteases, peptides, polypeptides, antibodies, antigens, antigenic epitopes, polynucleotides, hormones, carbohydrates, lipids, phospholipids, detection probes.

According to another preferred embodiment, the microarrays of the present invention comprise a plurality of chimeric proteins comprising a cellulose binding region, wherein said cellulose binding region is derived from a protein other than a member of a polysaccharidase complex. Particularly, the cellulose binding region is derived from the cellulosome of Clostridium thermocellum.

According to another preferred embodiment, the microarrays of the present invention comprise a plurality of chimeric proteins comprising a cellulose binding region, wherein said cellulose binding region is encoded by a polynucleotide comprising the nucleic acid sequence of SEQ ID NO 1 (Poole et al., FEMS Microbiol Lett. 1992, 78:181-6).

According to another preferred embodiment, the polynucleotide comprises a nucleic acid sequence having at least 50% homology, preferably 60-80% homology and more preferably about 90% homology to the nucleic acid sequence of SEQ ID NO 1.

According to yet another preferred embodiment, the polynucleotide further comprises at least one additional nucleic acid sequence selected from the group consisting of: SEQ ID NO 2, SEQ ID NO 3 or fragments thereof (Poole et al., ibid).

According to another preferred embodiment, the microarrays of the present invention comprise a plurality of chimeric polypeptides comprising a cellulose binding region, wherein said cellulose binding region comprises the amino acid sequence of SEQ ID NO 4 or cellulose binding fragments thereof (Morag et al., Appl. Environ. Microbiol. 1995, 61:1980-6).

According to another preferred embodiment, the cellulose binding region comprises an amino acid sequence having at least 50% homology, preferably about 60-80% homology, to the amino acid of SEQ ID NO 4.

According to another preferred embodiment, the cellulose binding region further comprises at least one additional amino acid sequence selected from the group consisting of: SEQ ID NO 5, SEQ ID NO 6 or fragments thereof (Morag et al., ibid).

It is to be understood explicitly that the scope of the present invention encompasses homologs, analogs, variants and derivatives, including shorter and longer polypeptides, proteins and polynucleotides, as well as polypeptide, protein and polynucleotide analogs with one or more amino acid or nucleic acid substitution, as well as amino acid or nucleic acid derivatives, non-natural amino or nucleic acids and synthetic amino or nucleic acids as are known in the art, with the stipulation that these variants and modifications must preserve the capacity of binding cellulose of the original molecule in the context of the microarray of the present invention. Specifically, any active fragments of the active polypeptide or protein as well as extensions, conjugates and mixtures are disclosed according to the principles of the present invention.

According to additional aspects, the present invention provides methods for fabricating the microarrays of the invention and methods of screening the microarrays of the invention.

According to another embodiment, the present invention provides a method for generating a microarray comprising a plurality of chimeric proteins comprising a cellulose binding region devoid of cellulase activity wherein the cellulose binding region further contains at least one exogenous peptide introduced therein while maintaining the capacity of binding a cellulose polymer, comprising the following steps:
(a) providing a polynucleotide encoding a cellulose binding region;
(b) introducing at least one oligonucleotide sequence encoding at least one exogenous peptide into the polynucleotide of (a) at a predetermined location;
(c) expressing the modified polynucleotide of (b) to obtain a chimeric protein comprising a cellulose binding region wherein the cellulose binding region further contains at least one exogenous peptide introduced therein;
(d) determining the binding efficiency of the chimeric protein of (c) to a cellulose polymer;
(e) attaching a plurality of chimeric proteins of (d) onto a solid support comprising a cellulose polymer to form an array of immobilized chimeric proteins;
(f) determining the exposure of the at least one exogenous peptide contained within the chimeric protein to the external environment at an accessible orientation.

According to another embodiment, the present invention provides a method for the production of an array comprising a plurality of chimeric proteins comprising a cellulose binding region devoid of cellulase activity, wherein each chimeric protein comprises at least one exogenous biologically active moiety fused to the cellulose binding region while maintaining the capacity to bind a cellulose polymer with high affinity and wherein the at least one biologically active moiety is exposed to the external environment in an accessible orientation while said protein is attached to a solid support, comprising the steps of:
(a) providing a polynucleotide comprising a nucleic acid encoding a cellulose binding region devoid of cellulase activity joined to an additional in frame sequence encoding an exogenous biologically active moiety;
(b) expressing the polynucleotide of (b) to obtain a fusion protein;
(c) determining the binding efficiency of the fusion protein of (b) to a cellulose polymer;
(d) attaching a plurality of chimeric proteins of (d) onto discrete locations over a solid support comprising a cellulose polymer to form an array of immobilized chimeric proteins; and
(e) determining the exposure of the at least one biological active moiety to the external environment at an accessible orientation.

According to some embodiments the modifications at the terminus sequence are random and according to other embodiments the modifications are deliberate.

According to another embodiment, the present invention provides a method for the production of microarray comprising a plurality of chimeric proteins comprising a cellulose binding region capable of binding cellulose polymers with high affinity, wherein each chimeric protein comprises at least one additional biologically active moiety covalently linked to the cellulose binding region while maintaining the capacity to bind a cellulose polymer with high affinity and wherein the at least one biologically active moiety is exposed to the external environment in an accessible orientation while said protein is attached to a solid support, comprising the steps of:
(a) providing a polynucleotide comprising a nucleic acid encoding a cellulose binding region devoid of cellulase activity;
(b) expressing the polynucleotide of (a) to obtain a protein comprising a cellulose binding region devoid of cellulase activity;
(c) covalently linking the protein of (b) to at least one biologically active moiety to obtain a chimeric protein;
(d) determining the binding efficiency of the chimeric protein of (c) to a cellulose polymer;
(e) attaching a plurality of chimeric proteins of (d) onto discrete locations over a solid support comprising a cellulose polymer to form an array of immobilized chimeric proteins; and
(f) determining the exposure of the at least one biological active moiety to the external environment at an accessible orientation.

According to a further embodiment, the present invention provides a method for screening a plurality of peptides for interactions with one or more components of a fluid sample, comprising the steps of:
(a) providing an array of discrete locations over a solid surface and a plurality of proteins immobilized at the discrete locations, wherein the surface comprises a cellulose polymer, therein the proteins comprise a cellulose binding region which is devoid of cellulase activity, wherein the cellulose binding region further contains at least one exogenous peptide introduced therein, to form an addressable array of locations each having one or more said proteins displayed within said location such that each exogenous peptide is exposed in an accessible orientation to a fluid sample in the external environment;
(b) contacting the array of (a) with a sample to be analyzed;
(c) detecting binding interactions between the array of (b) and the sample to be analyzed; and
(d) determining the loci of interaction.

According to a further embodiment, the present invention provides a method for screening a plurality of biologically active moieties for interactions with one or more components of a fluid sample comprising the steps of:
(a) providing a plurality of chimeric proteins immobilized at discrete locations over a solid surface, wherein the surface comprises a cellulose polymer, wherein the chimeric proteins comprise a cellulose binding region devoid of cellulase activity and at least one biologically active moiety fused or covalently linked thereto;
(b) forming an addressable array of the chimeric proteins of (a) at discrete locations, each having at least one biologically active moiety displayed at each said location such that the at least one biologically active moiety is exposed in an accessible orientation to the external environment;
(c) contacting the array of (b) with a sample to be analyzed;
(d) detecting binding interactions between the array of (b) and the sample to be analyzed; and
(e) determining the loci of interaction.

These and other aspects of the present invention will be more fully understood from the drawings, detailed description and examples which follows.

### BRIEF DESCRIPTION OF THE DRAWINGS

The invention will be more fully understood and further advantages will become apparent when reference is made to the following detailed description of the invention and the accompanying drawings in which:

FIG. 1 is the nucleotide sequence of the cellulose binding region (in bold; SEQ ID NO 1) and the flanking N-terminus (SEQ ID NO 2) and C-terminus linkers (SEQ ID NO 3) derived from Clostridium thermocellum and the corresponding amino-acid sequence of a cellulose binding region (SEQ ID NO 4) and the flanking N-terminus (SEQ ID NO 5) and C-terminus linkers (SEQ ID NO 6).

FIG. 2A illustrates the expression vector pET3d comprising SEQ ID NO 1, SEQ ID NO 2 and a variant of SEQ ID NO 3 comprising at the C-terminus a fragment of the nucleotide sequence encoding HIV antigen gp41.

FIG. 2B shows the amino-acid and nucleotide sequences of a fragment containing the mutated C-terminus linker (SEQ ID NO 3) comprising a fragment of the nucleotide sequence encoding gp41.

FIG. 3 shows ZEPHYRIN41 expression in total cell extract of BL1 (DE3) cells transfected with pET3d vector (lane 1) and in purified aliquots of the extracts (lanes 2-4).

FIG. 4 presents serum levels of HIV in infected and healthy individuals using recombinant gp41 and ZEPHYRIN41.

FIG. 5 exhibit fluorescence images reflecting levels of antibodies against HIV type 1 and type 2 antigens in sera of infected individuals using microarrays of the present invention comprising ZEPHYRIN36, ZEPHYRIN41 and ZEPHYRIN120 as compared to a control microarray (PBS).

FIG. 6A is an image of a microarray comprising a protein comprising the cellulose binding region of SEQ ID NO 4, labeled with Cy3, and fabricated on cellulose coated glass slide.

FIG. 6B is an image of a microarray comprising a biotinylated protein comprising the cellulose binding region of SEQ ID NO 4, detected by Streptavidin ALEXA FLOUR® 546 (Molecular Probes Europe BV, The Netherlands).

FIG. 6C is an image of a microarray comprising a protein comprising the cellulose binding region of SEQ ID NO 4, and labeled at the C-terminus linker with Cy5 labeled rabbit antibody directed against the protein.

FIG. 7A is an image of the fluorescence signals generated from the interaction between a microarray containing serial dilutions of non-purified (sonicate; right panel) and purified (left panel) protein comprising cellulose binding region of SEQ ID NO 4 and Cy5 labeled rabbit antibody directed against the protein of SEQ ID NO 4.

FIG. 7B is an image of a microarray containing His₆-tag labeled protein comprising the cellulose binding region of SEQ ID NO 4 diluted in PBS and in E. Coli cells extract (sonicate; right panel) and detected by Cy5 labeled rabbit His₆ tag antibodies directed against the protein of SEQ ID NO 4.

FIG. 8A presents an image of a microarray containing serial dilutions of biotinylated protein comprising the cellulose binding region of SEQ ID NO 4 fabricated on cellulose coated glass slide and detected by Streptavidin ALEXA FLOUR® 546.

FIG. 8B shows the linear regression analysis of the data from FIG. 8A.

FIGS. 9A-B is an image of a microarray of ZEPHYRIN41 contacted with sera from HIV type 1 infected individuals following incubation with Cy3 goat anti-human IgG (A) and with Cy5 rabbit directed against the cellulose binding region of SEQ ID NO 4.

FIG. 10 is an image of a microarray of serial dilutions in PBS of a protein comprising SEQ ID NO 4 wherein a fragment of the C-linker (SEQ ID NO 6) was modified to express the CS1 peptide, fabricated on cellulose-coated glass, following interaction with fluorescence-labeled Jurkat T-cells expressing a receptor recognizing the CS1 peptide.

FIGS. 11A-E illustrate microarrays of proteins comprising SEQ ID NO 4 chemically bound to biotin, following washing with various urea concentrations (0M, A; 1M, B; 3M, C; 6M, D), and the average fluorescence signal obtained from interacting the microarrays with streptavidin alexa fluor 546® (E).

FIGS. 12A-E show microarrays of proteins comprising SEQ ID NO 4 chemically bound to biotin, following washing with various SDS concentrations (0%, A; 0.5%, B; 1%, C; 3%, D) and the average fluorescence signal obtained from interacting the microarrays with streptavidin alexa fluor 546® (E).

FIGS. 13A-E present microarrays of proteins comprising SEQ ID NO 4 chemically bound to biotin, following washing with various Tween 20 concentrations (0%, A; 0.3%, B; 1.5%, C; 3%, D), and the average fluorescence signal obtained from interacting the microarrays with streptavidin alexa fluor 546® (E).

FIG. 14 illustrates a microarray of oligonucleotides conjugated to the Cysteine 55 of proteins comprising SEQ ID NO 4 (upper panel). The signal reflects interaction of complementary Cy5-oligonucleotide probe with the microarray.

FIG. 15A is a schematic description of the construct used to encode a microarray comprising a library of peptides.

FIGS. 15B-C present a scheme (B) and the corresponding image (C) of a peptide library microarray detected by Cy5-labeled rabbit anti SEQ ID NO 4.

FIG. 16A-C demonstrate a modification of SEQ ID NO 4 at loop 5/6 (A), the expression of a protein comprising this modification in induced BL21 cells (DE3; B) and molecular models of this proteins (C).

FIGS. 17A-B present fluorescence images of microarrays comprising proteins of SEQ ID NO 4 and fusion proteins (ZEPHYRIN-ZZ) detected by Cy5-rabbit IgG (A) and the corresponding linear regression analysis (B).

FIG. 18A demonstrates fluorescence images of microarrays comprising proteins having SEQ ID NO 4 biotinylated at the C-terminus following incubation with streptavidin.

FIGS. 18B-C show a fluorescence image of a microarray comprising lysate of cells expressing proteins comprising SEQ ID NO 4-linked to linear random peptides and visualized by Cy5-rabbit-anti-SEQ ID NO 4 (A) and the corresponding signal intensity per location (B).

### DETAILED DESCRIPTION OF THE INVENTION

A. Definitions

The term “a polypeptide comprising a cellulose binding region” as used herein refers to a polypeptide or a protein comprising an amino acid sequence having a substrate binding region of a polysaccharidase.

Preferably the cellulose binding region is obtained from Clostridium thermocellum or another microorganism that has essentially no cellulase activity. Cellulase is a hydrolase of cellulose and is capable of digesting cellulose.

The term “microarray” as used herein, refers to a solid support that provides a plurality of locations at which molecules may be bound. The number of different kinds of molecules bound at one location is small relative to the total number of different kinds of molecules in the microarray. In many embodiments, only one kind of molecule is bound at each feature. The term “array” is used interchangeably herein.

A “spatially addressable array” refers to an array in which the location of a molecule bound to the array can be recorded and tracked by its spatial address throughout any of the procedures carried out according to the invention. Spatial addressable arrays according to the invention can be one dimensional, for example a linear array; two dimensional; or three dimensional.

A particular position in an array is referred to as an “address” or “spot” or “locus”. Each address or spot has unique coordinates. The structure of a compound immobilized at a particular address or spot is definable by its coordinates.

By “immobilized on a solid support” or “attached to a solid support” is meant that a protein comprising a cellulose binding region, is attached to a solid surface, wherein the solid surface comprises a polymer capable of binding a cellulose binding region or an inert surface coated with such polymer in such a manner that the system containing the immobilized protein may be subjected to washing or other physical or chemical manipulation without being dislodged from that location.

The term “cellulose polymer” or “a polymer comprising cellulose” as used herein refers to homopolymers of cellulose comprising cellulose or modified cellulose, such as nitrocellulose, microcrystalline cellulose and etc., or to hetero-polymers which comprise various polysaccharides, in different ratios. The polysaccharides may be artificial or natural. Examples, without limitation, of cellulose polymers are cellulose acetate, microcrystalline cellulose, lignin, starch and xylane.

The term “exposed to the external environment” as used herein refers to peptide(s), particularly exogenous peptide(s) that are part of a polypeptide or a protein comprising a cellulose binding domain, wherein the peptide(s) are not covered by the structure the polypeptide or protein specifically when said polypeptide or protein is immobilized onto a solid support The uncovered immobilized peptide(s) are exposed in an accessible orientation.

A “library” refers to a collection of molecules, preferably bioactive moieties such as peptides, polypeptides, proteins, hormones or hormone precursors, carbohydrates, lipids, glycolipids, polynucleotides fusion proteins. The molecules may be naturally-occurring or artificially synthesized Molecules in the library are preferably spatially separated and bound to an array according to the invention.

A “bioactive” or “biologically active” moiety is any compound, either man-made or natural, that has an observable effect on a cell, a cell component or an organism. The observable effect is the “biological activity” of the compound.

The term “linker” or “native linker” as used herein refers to the C-terminus and/or the N-terminus sequence which flanks the cellulose binding region but has essentially no cellulose binding activity.

The term “variant” as used herein refers to a protein that possesses at least one modification compared to the original protein. Preferably, the variant is generated by modifying the nucleotide sequence encoding the original protein and then expressing the modified protein using methods known in the art. A modification may include at least one of the following: deletion of one or more nucleotides from the sequence of one polynucleotide compared to the sequence of a related polynucleotide, the addition of one or more nucleotides or the substitution of one nucleotide for another. Accordingly, the resulting modified protein may include at least one of the following modifications: one or more of the amino acid residues of the original protein are replaced by different amino acid residues, or are deleted, or one or more amino acid residues are added to the original protein. Other modification may be also introduced, for example, a peptide bond modification, cyclization of the structure of the original protein. A variant may have an altered binding ability to a cellulase substrate than the original protein. A variant may have at least 50% identity with the original cellulose binding region, preferably at least 60% or at least 70% identity.

The terms “polypeptide”, “peptide” and “protein” are used interchangeably to refer to polymers of amino acids of any length. These terms also apply to amino acid polymers in which one or more amino acid residues is an artificial chemical analogue of a corresponding naturally occurring amino acid, as well as to naturally occurring amino acid polymers. An amino acid polymer in which one or more amino acid residues is an “unnatural” amino acid, not corresponding to any naturally occurring amino acid, is also encompassed by the use of the terms “protein”, “peptide” and “polypeptide” herein.

As used herein, the terms “chimeric protein” as used herein refers to a modified protein, particularly a protein comprising a cellulose binding region and at least one exogenous biologically active moiety introduced therein or a fusion protein which comprises a cellulose binding region fused or covalently linked to at least one exogenous biologically active moiety. In preferred embodiments, the cellulose binding region is of SEQ ID NO 4 or variants thereof. In other preferred embodiments, the cellulose binding region further comprises at least one additional sequence selected from the group consisting of: SEQ ID NO 5 (corresponding to the N-terminus linker of SEQ ID NO 4), SEQ ID NO 6 (corresponding to the C-terminus linker of SEQ ID NO 4). In some embodiments of the present invention, the chimeric protein may be fused or joined to a second bioactive moiety.

“Fusion” refers to the joining together of a polynucleotide encoding a protein comprising a cellulose binding region and a polynucleotide encoding a biologically active moiety, in frame. Expression of the joint polynucleotides results in a chimeric protein also named hereinafter a “fusion protein”. The fusion protein of the present invention may comprise an enzymatic or chemical cleavage site upstream and preferably adjacent the N-terminus of the bioactive moiety and/or an enzymatic or chemical cleavage site downstream and preferably adjacent the C-terminus of the cellulose binding region thereby providing a means for recovering the bioactive moiety from the fusion protein through use of a cleaving agent.

“Covalently linking” refers to the binding of a protein comprising a cellulose binding region to a molecule using chemical modifications such as addition of bridging groups and the like, to facilitate the binding. Methods of covalently linking molecules are known by those skilled in the art.

Examples of biological active moieties include enzymes, such as nucleic acid modification enzymes, proteases, peptides, polypeptides, antibodies, antigens, antigenic epitopes and variants thereof, polynucleotides, hormones, carbohydrates, lipids, phospholipids, biotinylated probes.

The term “X% homology” is not intended to be limited to sequences having an X% homology over the entire length of the protein but is intended to include X% homology occurring in identified functional areas within the cellulose binding region and is also intended to include a functional area within the cellulose binding region which has the ability to bind cellulose with high affinity.

As used herein, the terms “label” or “labeled” refers to incorporation of a detectable marker, e.g., by incorporation of a radiolabeled amino acid or attachment to biotin moieties that can be detected by marked avidin (e.g. streptavidin containing a fluorescent marker or enzymatic activity that can be detected by optical or calorimetrically methods). Various methods of labeling molecules are known in the art and may be used. Examples of labels include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), biotinyl groups, predetermined epitopes recognized by a secondary reporter (e.g., leucine zipper pair sequences, binding sites for secondary antibodies, transcriptional activator polypeptide, metal binding domains, epitope tags).

“Nucleic acid” or “polynucleotide” refers to a nucleotide sequence comprising a series of nucleic acids in a 5′ to 3′ phosphate diester linkage that may be either an RNA or a DNA sequence. If the nucleic acid is DNA, the nucleotide sequence is either single or double stranded. A nucleic acid encoding the protein of the invention is RNA or DNA that encodes a protein capable of binding cellulose with high affinity, is complementary to nucleic acid sequence encoding such protein, or hybridizes to nucleic acid sequence encoding such protein and remains stably bound to it under stringent conditions.

As used herein, a “recombinant” nucleic acid or protein molecule is a molecule where the nucleic acid molecule which encodes the protein has been modified in vitro, so that its sequence is not naturally occurring, or corresponds to naturally occurring sequences that are not positioned as they would be positioned in a genome which has not been modified.

By “vector” is meant a polynucleotide molecule, preferably a DNA molecule derived, for example, from a plasmid, bacteriophage, or plant virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. The choice of the vector will typically depend on the compatibility of the vector with the host cell into which the vector is to be introduced. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin™) and the hph gene which confers resistance to the antibiotic hygromycin B. A vector according to the present invention is used for expression of the protein(s) comprising cellulose binding region. Thus, “an expression vector” refers to a vector containing nucleotide sequences containing transcriptional and translational regulatory information and such sequences are operably linked to nucleotide coding sequences.

As used herein, the term “operably linked” refers to a linkage in which the regulatory DNA sequences and the DNA sequence to be expressed are connected in such a way as to permit transcription and ultimately translation.

The term “host cell” refers to those cells capable of growth in culture and capable of expressing a protein or a fusion protein comprising a cellulose binding region. The host cells of the present invention encompass cells in vitro culture and include prokaryotic, eukaryotic, and insect cells. A host cell strain may be chosen which modulates the expression of the inserted sequences, or modifies and processes the gene product in the specific fashion desired. Expression from certain promoters can be elevated in the presence of certain inducers (e.g., zinc and cadmium ions for metallothionine promoters). Therefore expression of the protein or fusion protein of the invention may be controlled. The ability to control expression will be important if the protein or fusion protein is lethal to a host cell. Modifications (e.g., phosphorylation) and processing (e.g., cleavage) of protein products are important for the function of the protein. Different host cells have characteristic and specific mechanisms for the post-translational processing and modification of protein. Appropriate cell lines or host systems can be chosen to ensure the correct modification and processing of the protein or fusion protein expressed. Preferably, the host cell should secrete minimal amounts of proteolytic enzymes.

B. Preferred Modes for Carrying Out the Invention

(i) Cloning and Expression of Proteins Comprising Cellulose Binding Regions

The present invention provides microarrays comprising a plurality of proteins comprising a cellulose binding region and a biologically active moiety, capable of attaching to a cellulase substrate with high affinity.

In certain embodiments, the microarrays of the present invention comprise a plurality of proteins comprising cellulose binding region wherein the cellulose binding region is modified such that it contains at least one exogenous peptide sequence introduced therein while maintaining the capacity to bind cellulase substrates with high affinity.

The proteins may be prepared by transforming into a host cell a DNA construct comprising a DNA encoding a cellulose binding region wherein said region further contains at least one DNA fragment encoding a exogenous peptide of interest and growing the host cell to express the protein.

Preferably, the exogenous peptides comprise a sequence of 5 to 50 amino acid residues, more preferably a sequence of about 15 to 30 amino acid residues and most preferably a sequence of about 8 to 15 amino acids.

The modifications of the cellulose binding regions are introduced at predetermined sites such that the modified proteins maintain the ability to bind cellulose with high affinity. Moreover, modifications are introduced at sites which will be exposed to the external environment of the microarray upon attachment of the modified proteins to a solid support. Examples of sites which may be selected for modification in a cellulose binding region derived from Clostridium Thermocellum (i.e. SEQ ID NO 4) are: loop 5/6 located between β-strand 5 and β-strand 6, a loop between β-strand 7 and β-strand 8, a loop between β-strand 8 and β-strand 9, a loop between β-strand 3 and β-strand 4, the residues that form a second conserved site located in a shallow groove opposite to the cellulose-binding surface.

In other embodiments, the microarrays of the present invention comprise a plurality of fusion proteins comprising a cellulose binding region capable of binding polymers comprising cellulose with high affinity, wherein each fusion protein further comprises at least one biologically active moiety while maintaining the capacity to bind polymers comprising cellulose with high affinity. The biologically active moiety may be selected from the group consisting of peptides, proteins, polypeptides, polynucleotides, hormones, lipids and carbohydrates.

Fusion proteins comprising protein-based moieties may be prepared by transforming into a host cell a DNA construct comprising a DNA fragment encoding a cellulose binding region and the peptide of interest and growing the host cell to express the protein.

In yet other embodiments, the microarrays of the present invention comprise a plurality of chimeric proteins comprising a cellulose binding region and at least one biologically active moiety covalently linked to the cellulose binding region. Biologically active moieties include, by way of example and not limitation, polynucleotides, hormones, carbohydrates, lipids, phospholipids, biotinylated probes and the like.

In another embodiment, the microarrays of the present invention comprise a plurality of proteins comprising a cellulose binding region and at least two biologically active moieties. In some embodiments, the at least two biologically active moieties may be the same and in other embodiments the biologically active moieties may be different from each other.

In those embodiments where the at least two biologically active moieties are different, the microarray is particularly useful for detecting biological interactions between ligands and receptors which are mediated by a third component For example, the interaction of growth factors and their receptors may involve additional necessary components, as in the case of fibroblast growth factor (FGF) binding to FGF receptor that is mediated by both a protein-protein interaction and a protein-oligosaccharide interaction. Production of a suitable microarray requires exposure of the at least two biologically active moieties at each locus. In other words, the at least two biologically active moieties at each locus are simultaneously accessible to the external environment of the microarray upon attachment of the proteins comprising the cellulose binding region to a cellulase substrate.

We now disclose that unexpectedly simultaneous display of at least two different biologically active moieties at each locus of the microarrays of the present invention causes no mutual interference, as exemplified hereinbelow by a microarray comprising two different antibodies which recognize two different epitopes at each locus.

The nucleic acid encoding the protein(s) of the present invention includes nucleic acid of genomic, cDNA, synthetic, and semi-synthetic origin which, by virtue of its origin or manipulation, may be linked to a polynucleotide other than that to which it is linked in nature, and includes single or double stranded polymers of ribonucleotides, deoxyribonucleotides, nucleotide analogs, or combinations thereof, as long as the protein being encoded retains the ability to bind cellulose with high affinity. The nucleic acid encoding the protein of the present invention also includes various modifications known in the art, including but not limited to radioactive and chemical labels, methylation, caps, internucleotide modifications such those with charged linkages (e.g., phosphorothioates, phosphorodithioates, etc.) and uncharged linkages (e.g., methyl phosphonates, phosphotriesters, phosphoramidites, carbamites, etc.), as well as those containing pendant moieties, interchelators, chelators, etc. as long as the protein encoded by the nucleic acid retains the ability to bind cellulose with high affinity and in a reversible manner.

The nucleic acid encoding a protein comprising a cellulose binding region may be obtained from a variety of cell sources that produce a protein comprising cellulose binding regions that bind with high affinity and in a reversible manner or that produce mRNA encoding such protein. The preferred source of a nucleic acid encoding a protein comprising a cellulose binding region is Clostridium Thermocellum.

In another preferred embodiment, the present invention utilizes a protein comprising a cellulose binding region wherein the cellulose binding region is encoded by a polynucleotide comprising the nucleic acid sequence of SEQ ID NO 1 or fragments thereof. The fragments encode proteins capable of binding cellulase substrates, even when a sequence of an exogenous protein is introduced therein.

According to another preferred embodiment, the polynucleotide comprises a nucleic acid sequence having at least 50% homology, preferably 60-80% homology and more preferably about 90% homology to the nucleic acid sequence of SEQ ID NO 1.

Optionally, the nucleic acid sequence encoding a cellulose binding region may further comprises at least one of the native linkers flanking the N/C-termini of the cellulose binding region. Thus, nucleic acid sequence encoding a protein comprising a cellulose binding region comprising the nucleic acid sequence of SEQ ID NO 1 or fragments thereof may further comprise at least one additional nucleic acid sequence selected from the group consisting of: SEQ ID NO 2 (corresponding to the N-terminus linker of SEQ ID NO 1), SEQ ID NO 3 (corresponding to the C-terminus linker of SEQ ID NO 1).

It is now disclosed for the first time that a protein comprising a cellulose binding region including at least one of the native flanking sequences or linkers of said region provides unexpectedly high levels of expression with respect to the expression of said protein without said linker.

In other preferred embodiments, the microarrays of the present invention comprise a plurality of fusion proteins comprising an amino acid sequence of SEQ ID NO 4. Optionally, the microarrays of the present invention may comprise a fusion protein comprising a cellulose binding region of an amino acid sequence having at least 50% homology, preferably about 60-80% homology, to the amino acid of SEQ ID NO 4.

Preferably, the microarrays of the present invention comprise a plurality of fusion proteins comprising an amino acid sequence of SEQ ID NO 4 and further comprising at least one additional amino acid sequence selected from the group consisting of: SEQ ID NO 5, SEQ ID NO 6.

It is to be understood explicitly that the scope of the present invention encompasses homologs, analogs, variants and derivatives, including shorter and longer proteins and polynucleotides, as well as protein and polynucleotide analogs with one or more amino acid or nucleic acid substitution, as well as amino acid or nucleic acid derivatives, non-natural amino or nucleic acids and synthetic amino or nucleic acids as are known in the art, with the stipulation that these modifications must preserve the capacity of binding cellulose of the original molecule in the context of the microarray of the present invention. Specifically any active fragments of the active protein as well as extensions, conjugates and mixtures are disclosed according to the principles of the present invention.

A preferred strategy for generating fusion proteins comprising biologically active moieties is by introducing random or deliberate mutations within at least one of the linkers which flank the cellulose binding region.

Mutations in a nucleic acid sequence can be created through site-directed mutagenesis, also termed herein “deliberate mutagenesis”, or through random mutagenesis as known in the art Random mutagenesis may be generated by error-prone PCR including the use of polymerase of increased mutagenic activity, the addition of disproportional amount of a given nucleic acid and the like. Another useful procedure for performing such mutagenesis is called “DNA shuffling” (see Harayama, S., Trends Biotechnol., 1998, 16:76-82). For generating a library of peptides the C-terminus linker or the N-terminus linker of the protein of the present invention may be randomly mutated. Preferably, the resulting library of peptides is immobilized onto a solid surface comprising a polymer comprising cellulose such that the various peptides are exposed, and preferably oriented so as to be accessible to the external environment or medium while the cellulose binding region is attached to said solid surface.

Site-directed mutagenesis of specific nucleotides can be used to create mutant linkers with particular properties using methods such as a closing oligonucleotide method described previously (Slilaty et al., 1990, Anal. Biochem. 185:194-200) and mutagenic oligonucleotide. Oligonucleotides may be synthesized by standard methods known in the art, e.g. by use of an automated DNA synthesizer (such as are commercially available from Biosearch, Applied Biosystems, etc.). In some preferred embodiments, the site-directed mutagenesis is at the C-terminus linker (e.g. SEQ ID NO 3 encoded by SEQ ID NO 6) wherein the polynucleotide sequence encoding the C-terminus linker is modified such that it encodes a fragment of an HIV type 1 or type 2 antigen such as gp36 (for UV type 2) resulting in a protein called herein ZEPHYRIN36, gp41 (for HIV type 2) resulting in a protein called herein ZEPHYRIN41, gp120 (for HIV type 1) resulting in a protein called herein ZEPHYRIN120; in other preferred embodiment of the present invention, the C-terminus linker (SEQ ID NO 3) is mutated to express a ZZ domain protein (Nord et al., Protein Eng. 1995 8:601) resulting in a protein called herein ZEPHYRIN-ZZ.

The nucleic acid encoding the protein(s) of the present invention may be used to construct recombinant expression vectors capable of expressing the protein(s). In constructing an expression vector for a fusion protein, the nucleic acid encoding the protein comprising a cellulose binding region will be linked or joined to the nucleic acid encoding the biologically active moiety such that the open reading frame of the protein comprising a cellulose binding region and the biologically active moiety is intact, allowing translation of the fusion protein to occur.

Many vectors are available, and selection of the appropriate vector will depend on whether it is to be used for nucleic acid amplification or for nucleic acid expression, the size of the nucleic acid to be inserted into the vector, and the host cell to be transformed with the vector. Transfection of the host cell can be effected in a number of ways well known to those of ordinary skill in the art, including, but not limited to, electroporation, injection, calcium chloride precipitation and retroviral introduction. Furthermore, the nucleic acid can be either integrated with the genome of the hose cell or not.

Each vector contains various components depending on the function (amplification of nucleic acid or expression of nucleic acid) and the host cell for which it is compatible.

The preferred host cell for cloning and expression of the proteins of the present invention is a prokaryotic cell. Prokaryotes are particularly useful for rapid production of large amounts of nucleic acid, for production of single-stranded nucleic acid templates used for site-directed mutagenesis, for screening many mutants simultaneously, and for nucleic acid sequencing of the mutants generated. An example of a prokaryotic cell useful for cloning and expression of the proteins of the present invention is E. Coli strain BL21 (DE3; Novagen, WI, USA).

Various expression vector/host systems may be utilized equally well by those skilled in the art for the recombinant expression of proteins of the present invention. Such systems include but are not limited to microorganisms such as bacteria transformed with recombinant bacteriophage DNA, plasmid DNA or cosmid DNA expression vectors containing the desired coding sequence; yeast transformed with recombinant yeast expression vectors containing the desired coding sequence; insect cell systems infected with recombinant virus expression vectors (e.g., baculovirus) containing the desired coding sequence; plant cell systems infected with recombinant virus expression vectors (e.g., cauliflower mosaic virus CaMV; tobacco mosaic virus, TMV) or transformed with recombinant plasmid expression vectors (e.g., Ti plasmid) containing the desired coding sequence; or animal cell systems infected with recombinant virus expression vectors (e.g. adenovirus, vaccinia virus) including cell lines engineered to contain multiple copies of the desired nucleic acid either stably amplified (e.g., CHO/dhfr, CHO/glutamine synthetase) or unstably amplified in double-minute chromosomes (e.g., murine cell lines).

Vector components generally include, but are not limited to, one or more of the following: a signal sequence, an origin of replication, one or more marker genes, an enhancer element, a promoter, and a transcription termination sequence. The expression elements of these vectors vary in their strength and specificities. Depending on the host/vector system utilized, any one of a number of suitable transcription and translation elements may be used. For example, when cloning in prokaryotic cell systems, promoters isolated from the genome of prokaryotic cells, (e.g., the bacterial tryptophan promoter) may be used. Promoters produced by recombinant DNA or synthetic techniques may also be used to provide for transcription of the inserted sequences.

A signal sequence may be a component of the vector, or it may be a part of the nucleic acid, encoding the proteins of the invention, that is inserted into the vector. The signal sequence may be the naturally occurring sequence or a non-naturally occurring sequence. The signal sequence should be one that is recognized and processed by the host cell. An origin of replication refers to the unique site of initiation of replication of a host organism. It is desirable for cloning and expression vectors to comprise a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics or other toxins, e.g. ampicillin; complement auxotrophic deficiencies; or supply critical nutrients not available from complex media. One example of a selection scheme utilizes a drug to arrest growth of a host cell. Those cells that are successfully transformed with a heterologous gene express a protein conferring drug resistance and thus survive the selection regimen. Expression vectors used in prokaryotic host cells will also contain sequences necessary for the termination of transcription and for stabilizing the mRNA.

Construction of suitable vectors containing one or more of the above listed components and including the desired coding and control sequences employs standard ligation techniques. Isolated plasmids or nucleic acid fragments are cleaved, tailored, and religated in the form desired to generate the plasmids required.

Host cells are transfected and preferably transformed with the above-described expression or cloning vectors of this invention and cultured in conventional nutrient media modified as appropriate for inducing promoters, selecting transformants, or amplifying the genes encoding the desired sequences. An advantage of the proteins of the present invention is the simplicity of their production. The proteins of the invention are secreted to the growth medium or preferably accumulate in a soluble form within the cell. The proteins of the present invention do not tend to form inclusion bodies in the cytoplasm of E. Coli like for example proteins comprising a cellulose binding domain derived from Clostridium Cellulovorans.

(ii) Microarray Fabrication and Application

The microarrays of the present invention comprise a plurality of proteins comprising a cellulose binding region attached to a solid phase support comprising a cellulase substrate. The proteins are immobilized on a solid surface, preferably in a spatially addressable manner. Thus, fabrication of the microarrays of the present invention involves the step of applying said proteins onto a predefined location a surface, preferably a solid surface, comprising a polymer comprising cellulose.

Fabrication of the microarrays of the present invention is simple and straightforward due to the following advantages:
(a) The microarrays of the present invention may consist essentially of cellulose polymers and a plurality of proteins comprising a cellulose binding region;
(b) Attachment of the proteins comprising a cellulose binding region to the solid support of the microarrays of the present invention does not require any chemical modification, due to the high affinity of the cellulose binding region to cellulose.
(c) Fabrication of the microarrays of the present invention does not require purification of the proteins comprising a cellulose binding region prior to their attachment onto cellulose polymers.

The nature and geometry of the solid surface will depend upon a variety of factors, including, among others, the type of array (e.g., one-dimensional, two-dimensional or three-dimensional). Generally, the surface can be composed of any material which will permit immobilization of the proteins and which will not melt or otherwise substantially degrade under the conditions used in the applications of the array.

A number of materials suitable for use as surfaces in the instant invention have been described in the art. Exemplary suitable materials include cellulose homopolymers and cellulose heteropolymers, selected from cellulose acetate, microcrystalline cellulose, lignin, starch, xylane.

The solid surface may be in the form of beads, particles or sheets, and may be permeable or impermeable, depending on the type of array, wherein the surface is coated with a suitable material enabling binding of cellulose binding region at high affinity. The solid phase support may further comprise a wide variety of compositions, while maintaining the ability to bind a cellulose binding region at high specificity. For example, for linear or three-dimensional arrays the surface may be in the form of beads or particles, fibers (such as glass wool or other glass or plastic fibers) or glass or plastic capillary tubes. For two-dimensional arrays, the solid surface is preferably in the form of plastic, micromachined chips, membranes, slides, plates or sheets in which at least one surface is substantially flat, wherein these surfaces may comprise glass, plastic, silicon, low cross-linked and high cross-linked polystyrene, silica gel, polyamide, and the like. Preferred solid surface for use with the microarrays of the present invention include, by a way of a non-limiting example, two- are glass slides coated with cellulose.

The arrays of the present invention may be of any desired size. The upper and lower limits on the size of the array are determined solely by the practical considerations of resolution, size of molecules expressed at each address and the like.

Either a population of discrete proteins is employed to form the array, such that each address presents a different molecule, or a single or a few addresses are employed with a similar protein. Indeed, in many applications, redundancies in the spots are desirable for the purposes of acting as internal controls.

In some embodiments, the present invention provides microarrays consisting essentially cellulose and a plurality of proteins comprising a cellulose binding region. Fabrication of such microarrays is simple and does not require additional steps such as coating a given surface with cellulase substrate or intermediate layers such as adhesion layers.

Technologies allowing the deposition of droplets containing proteins comprising cellulose binding regions and biologically active moieties onto a suitable solid surface are known in the art. An ink-jet printing technology for deposition of small droplets while avoiding overlap or splatter is disclosed in U.S. Pat. No. 5,449,754. This technology is particularly effective for creating peptide microarrays. Any other contact spotter or arrayer that deposits droplets comprising proteins comprising a cellulose binding region onto a solid surface comprising cellulose polymer are suitable for fabricating the microarrays of the present invention.

According to another embodiment, the present invention provides a method for generating a microarray comprising a plurality of proteins comprising a cellulose binding region wherein the cellulose binding region further containing at least one biologically active moiety introduced therein while maintaining the capacity of binding polymer comprising cellulose, comprising the following steps:
(a) providing a polynucleotide encoding a cellulose binding region;
(b) introducing at least one oligonucleotide sequence encoding at least one exogenous peptide into the polynucleotide of (a) at a predetermined location;
(c) expressing the modified polynucleotide of (b) and harvesting the resulting polypeptide; and
(d) determining the binding efficiency of the modified polypeptide of (c) to a cellulase substrate;
(e) immobilizing a plurality of proteins of (c) onto a solid support comprising a polymer comprising cellulose to form an array of proteins; and
(f) determining the exposure of the at least one exogenous peptide to the external medium.

An exogenous DNA sequence encoding an exogenous peptide may be introduced into any site within the cellulose binding region providing that the ability of the region to bind cellulose polymers is retained. Particularly suitable sites within a cellulose binding region comprising SEQ ID NO 4 are: loop 5/6 between β-strand 5 and β-strand 6, a loop between β-strand 7 and β-strand 8, a loop between β-strand 8 and β-strand 9, a loop between β-strand 3 and β-strand 4, the residues that form a second conserved site located in a shallow groove on the opposite side of the cellulose-biding surface.

According to another embodiment, the present invention provides a method for the production of microarrays comprising a plurality of fusion proteins comprising a cellulose binding region capable of binding cellulose polymers with high affinity, wherein each fusion protein comprises at least one biologically active moiety while maintaining the capacity to bind polymer comprising cellulose with high affinity and wherein the at least one biologically active moiety is exposed to the external environment while said protein is attached to a solid support, comprising the steps of:
(a) providing a polynucleotide comprising a nucleic acid encoding a cellulose binding region flanked by an N-terminus or a C-terminus sequence that has essentially no cellulose binding activity;
(b) modifying the flanking terminus sequence of the nucleic acid of (a) such that the modified terminus sequence comprises at least one additional sequence encoding an exogenous biological active moiety;
(c) expressing the modified polynucleotide of (b) and harvesting the resulting fusion protein;
(d) determining the binding efficiency of the modified protein of (c) to a polymer comprising cellulose;
(e) immobilizing a plurality of proteins of (d) onto a solid support comprising a cellulase substrate to form an array of fusion proteins; and
(f) determining the exposure of the biological active moiety to the external environment.

According to another embodiment, the present invention provides a method for the production of microarray comprising a plurality of chimeric proteins comprising a cellulose binding region capable of binding cellulose polymers with high affinity, wherein each chimeric protein comprises at least one additional biologically active moiety covalently linked to the cellulose binding region while maintaining the capacity to bind a cellulose polymer with high affinity and wherein the at least one biologically active moiety is exposed to the external environment in an accessible orientation while said protein is attached to a solid support, comprising the steps of:
(a) providing a polynucleotide comprising a nucleic acid encoding a cellulose binding region devoid of cellulase activity;
(b) expressing the polynucleotide of (b) to obtain a protein comprising a cellulose binding region devoid of cellulase activity;
(c) covalently linking the protein of (b) to at least one biologically active moiety to obtain a chimeric protein;
(d) determining the binding efficiency of the chimeric protein of (c) to a cellulose polymer,
(e) attaching a plurality of chimeric proteins of (c) onto discrete locations over a solid support comprising a cellulose polymer to form an array of immobilized chimeric proteins; and
(f) determining the exposure of the at least one biological active moiety to the external environment at an accessible orientation.

According to some embodiments the modifications at the terminus sequence are random and according to other embodiments the modifications are deliberate.

According to a further embodiment, the present invention provides a method for screening a sample, comprising the steps of:
(a) providing an addressable microarray comprising a plurality of chimeric proteins comprising a cellulose binding region and at least one biological active moiety wherein each protein is immobilized onto a solid phase support by attachment to a cellulase substrate comprised within said support and each biological active moiety is exposed to the external environment;
(b) contacting the microarray of (a) with a sample to be analyzed;
(c) detecting binding interactions between the microarray of (b) and the sample to be analyzed; and
(d) determining the loci of interaction.

For fabricating the arrays of the present invention, it is not required to purify the proteins comprising cellulose binding regions prior to their attachment to a support comprising cellulase substrate, as exemplified hereinbelow. This property of the microarrays of the present invention is particularly practical for the fabrication of libraries containing a large number of proteins.

In order to conduct microarray assays, the non-immobilized component that is a sample of interest is added to the microarray comprising a coated surface containing the anchored components of the microarray. Following contact of the microarray of the present invention with a sample of interest, the array is optionally washed, typically under conditions such that any complexes formed will remain immobilized on the solid surface and unbound material will be removed.

The detection of complexes anchored on the solid surface can be accomplished in a number of ways. In some embodiments, the non-immobilized sample is pre-labeled, and the detection is directed for label immobilized on the surface indicating that complexes were formed. In other embodiments, the non-immobilized sample is not pre-labeled, an indirect label can be used to detect complexes anchored on the surface; e.g., using a labeled antibody specific for the previously non-immobilized sample (the antibody, in turn, may be directly labeled or indirectly labeled with a labeled anti-Ig antibody). In another preferred embodiment, the immobilized molecules of the microarray are labeled, the array can be scanned or otherwise analyzed for detectable assay signal, and the signal from each labeled spot, or alternatively from all spots, quantified.

A critical feature of a direct detection of the array of the invention is the presence of an amount of a label at each position within the array that is proportional to the amount of molecule immobilized at that particular spot. Thus, it is important that the efficiencies of the coupling reactions which are used to immobilize the labeled molecules are substantially similar.

Virtually any label that produces a detectable, quantifiable signal and that is capable of being attached to an immobilized protein comprising a cellulose binding region on a substrate can be used in conjunction with the array of the invention Suitable labels include, by way of example and not limitation, radioisotopes, fluorophores, chromophores, chemiluminescent moieties, etc. In embodiments where the label is attached to a polynucleotide, the label can be attached to any part of the polynucleotide, including the free terminus or one or more of the bases. Preferably, the position of the label will not interfere with interaction between a desired sample and the immobilized molecules and with the detection in case of an interaction between the desired sample and an immobilized molecule of the array. Suitable methods of making labeled molecules are well known in the art Preferred labels for direct detection of an array according to the principle of the present invention are: biotin.

In the case where each spot in the arrays of the invention contains an amount of a label or “tracer” proportional to the amount of molecules immobilized at the particular spot, the signals obtained from the arrays of the invention can be normalized. As a consequence, signal intensities from spots within a single array, or across multiple arrays, can be directly compared. A normalized signal of a particular spot may be defined by (Iₜ−I₀)/I₀, where Iₜ is the intensity of the signal of the spot after contacting with a sample of interest and I₀ is the intensity of the background signal of the spot before contacting with a sample of interest.

Various methods and devices for detection and analysis of the microarray of the present invention are known in the art. Practically, any imaging system that is capable of detecting with a resolution appropriate to the size of the array features can be utilized. For example, a method for screening an array of proteins for interactions with a fluid sample is disclosed in U.S. Pat. No. 6,475,809. Imaging apparatus may be selected, without any limitation, from ScanArray 4000 (General Scanning), Biochip Imager (Hewlett Packard), GMS 418 Array Scanner (Genetic Microsystems), GeneTAC 1000 (Genomic Solutions), Chip Reader (Virtek). Phosphorimager systems are available for detecting radiolabels, e.g. Cyclone (Packard Instrument company) and BAS-5000 (Fujifilm).

The microarrays and the methods of the present invention are particularly suitable for use for the fabrication of libraries such as peptide, antibody, DNA and chemical libraries. The advantage of the microarray of the present invention over the existing technologies is that it enables to produce a functional microarray with a library of non purified components, such as peptides and proteins, as exemplified hereinbelow, and hence provides an ideal platform for applications which require a large workload at high speed, high sensitivity and low cost.

The arrays of the invention are useful in assays employing unlabeled immobilized molecules and further employing interaction with labeled targets or interaction with non-labeled target following an interaction with a secondary labeled target. A main requirement in such assays is that a detectable signal at spots occurs only upon recognition/interaction between an immobilized molecule of the microarray of the invention and the sample of interest.

The microarrays of the present invention may be utilized in immuno-assays for detecting the presence of substances of interest in a test sample. An immunoassay using the microarrays of the invention may comprise the following steps: (a) contacting a test sample with a microarray of the invention comprising (i) a protein comprising a cellulose binding region capable of binding to cellulose with high affinity, and (ii) a library of second molecules, (iii) a detectable label capable of binding a substrate of interest or the cellulose binding protein of the microarray, and (iv) a solid support comprising cellulase substrate; and (b) separating the microarray with the bound substrate(s) from unbound components; and (c) incubating the microarray of step (b) with a sufficient amount of a detectable label, the label capable of binding to a substance of interest; and (d) separating the microarray of step (c) from unbound components and determining the presence or absence of the label, to provide an indication of the presence or absence of the substance of interest in the test sample.

Having now generally described the invention, the same will be more readily understood through reference to the following examples, which are provided by way of illustration and are not intended to be limiting of the present invention.

### EXAMPLES

Materials and Methods

Lysate biotinamidocaproate n-hydroxysucciniimide ester, isopropyl β-d-thiogalactopyranoside was purchased from SIGMA Chemical Co., St. Louis, Mo., USA. Peroxidase-conjugated rabbit anti-human IgG, peroxidase-conjugated rabbit anti-human IgA, peroxidase-conjugated rabbit anti-human IgM and Cy3 goat anti human IgG was from Jackson Immunoresearch, Inc., PA, USA. Cyto 82 were from molecular probes, Eugene, Oreg., USA. Mouse anti his6-tag was from Serotec Ltd, Oxford, UK. T7 RNA polymerase was from Novagen, Madison, Wis., USA. (sulfosuccinimidyl 4-[n-maleimmidomethyl]-cyclohexane-1-carboxylate) (Sulfo-SMCC) was purchased from Pierce, Rockford, Ill., USA. 5′-end amino modified oligonucleotide and 5′-end Cy5 oligonucleotide probes were synthesized by IDT, inc. Coralville, Iowa, USA.

The principles of the invention are exemplified hereinbelow using a microarray which comprises a protein comprising a cellulose binding region derived from Clostridium Thermocellum. It is explicitly intended that the microarrays and methods of the invention are applicable to a wide variety of proteins comprising a cellulose binding region.

Protein Purification by Cellulose Affinity Chromatography

Bacterial cells (100 ml of induced culture) from BL21 (DE3) carrying plasmid pET3d-ZEPHYRIN36 and induced for 16 hr with 0.1 mM IPTG were centrifuged and the pellet was suspended in 10 ml and disrupted by sonication, 40% amplitude, 5 cycles of 2 min each). Total cell extract was centrifuged at 15,000 g for 15 min at 4° C., and the soluble fraction was collected. Aliquots of 1 gram of microcrystalline cellulose was added into the soluble fraction and the mixture was maintained for 1 hr at room temperature under continuous stirring following centrifugation at 10,000 g for 5 min at 4° C. The supernatant was discarded and the cellulose pellet was resuspended and washed with 40 ml PBS containing 1M NaCl. This washing procedure, with high salt concentration, was repeated twice and was followed by two sequential washing steps with PBS. The ZEPHYRIN36 was eluted from the cellulose by the addition of 5 ml 1% triethylamine. The eluted soluble protein was neutralized immediately by addition of 0.5 ml of 1M phosphate buffer pH 5.6. The purified protein was divided into small aliquot and stored at −20° C.

Protein Purification by High Temperature Incubation

Bacterial cells (100 ml of induced culture) from BL21 (DE3) carrying plasmid pET3d-ZEPHYRIN36 and induced for 16 hr with 0.1 mM IPTG were centrifuged and the pellet was suspended in 10 ml and with 0.1 mM IPTG were centrifuged and the pellet was suspended in 10 ml and disrupted by sonication. The total cell extract was centrifuges at 15,000 g for 15 min at 4° C., and the soluble fraction was collected and saved. The soluble fraction was transferred into 70° C. water bath and allowed to incubate for 60 min. The sample was then centrifuged at 10,000 g for 5 min at 4° C. The purified protein was divided into small aliquot and stored at −20° C.

Preparation of Microarrays

Double-sided adhesive tape was used for casting the plain glass slide (75 mm×25 mm) with various membranes. The casting of glass slide comprises two steps. In the first step, one side of the tape was used to stick on the tape on the glass slide and followed by exposing the second surface of the adhesive tape. In the second step, cellulose membrane (50 mm×18 mm) was carefully stuck on the exposed side. This step was accomplished by tightly pressing the membrane on the adhesive side using glass pipette. The cellulose membranes were prepared from either regenerated cellulose or from cellulose acetate.

A contact-printing manual spotter (MicroCaster slide microarrayer, Schleicher & Schuell Inc, NH, USA), equipped with 8 pin tool of 0.2 mm diameter, was used to apply spots of the protein samples in an ordered array onto the cellulose-coated glass slides. The spot-to-spot distance was about 0.2 mm and droplet volume was 2 nL.

Samples of proteins comprising a cellulose binding region, having the amino acid sequence of SEQ ID NO 4 were serially diluted in PBS or in crude E. Coli extract, before spotting. After fabrication the slides were allowed to dry at room temperature for 30 min and then either used immediately or stored in a 50-ml polypropylene tube, stored at 4° C.

Assay Performance and Detection System

Fabricated microarray slides were removed from the 50-ml and were transferred into polypropylene slide container. The slides were then blocked with 2% BSA in PBS (4 ml per slide). After 60-min incubation at room temperature, the blocking solution was removed and residual solution was shaken off by tapping the slide over filter paper. Without allowing the microarray to dry a 4-ml aliquot of appropriate legend in 2% BSA/PBS was then added and allowed to react for 1 hr at room temperature under continuous mixing. The microarray slide was washed extensively with PBS/0.1% Tween-20 (five times, 4 min each) and if needed was reacted for additional hour with appropriate secondary antibody. After another washing step fluorescence signals measured using scan arrayer (ScanArray Lite, Perkin Elmer, Mass., USA.).

Cell Adhesion ASSAY

Jurkat T-cells, a human leukemia line, were grown under the appropriate growing conditions. Shortly before the adherence assay, cells were washed with 5 mM EDTA in Tris-buffered saline (TBS, 10 mM Tris-HCl, pH 7.4, 150 mM NaCl) and then incubated with Cyto 82 (2 μM final concentration) for 30 min under continuous agitation. Labeled cells were washed twice with the same buffer and applied directly on the fabricated microarray slides.

Preparation of Crude E. Coli Extract

One liter of E. Coli culture, containing unrelated plasmid was centrifuged, and resuspended in 40 ml PBS. The cells were then sonicated on ice and centrifuged again. Supernatant was collected and diluted 4 times with 2% BSA in PBS. Small aliquots were stored at −20° C.

Protein Biotinylation and Cy5 Labeling

Purified protein, which contains the amino acid sequence of SEQ ID NO 4, was biotinylated, using biotinamidocaproate N-hydroxysuccinimide ester or biotin maleimide at a 5-fold molar ratio of reagent to protein as described previously (2). Rabbit anti SEQ ID NO 4, XylanseT6Ce1S-dockerin and mouse anti-his₆-tag were labeled with Cy5 according to the manufacturer instructions.

ELISA Test

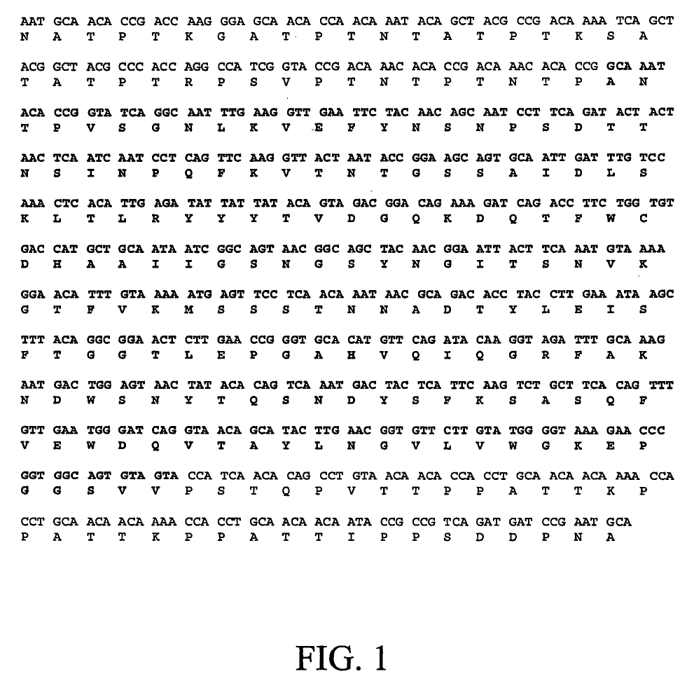

96-well microtiter plates (Maxisorp, Nunc) were coated by overnight incubation at 4° C. with a recombinant gp41 (1 μg/ml), a protein having SEQ ID NO 4 (5 μg/ml) and ZEPHYRIN41 fusion protein comprising both the recombinant gp41 and SEQ ID NO 4 (5 μg/ml) dissolved in 0.1M NaHCO₃, pH8.1 (100 μl/well). The plates were washes once with PBS and blocked with 2% BSA/PBS (200 μl/well) for 1 hr at room temperature. Aliquots of 200 μl/well of healthy and HIV type 1 infected individuals serum diluted 1:100000 with 2% BSA/PBS were added to the wells and the plate was incubated for 1 hr at room temperature. The plates were washed subsequently 5 times with PBS/Tween20—0.1% and peroxidase-conjugated rabbit anti-human IgG, peroxidase-conjugated rabbit anti-human IgA and peroxidase-conjugated rabbit anti-human IgM diluted 1:10000 in 2% BSA/PBS was then applied to the wells for 1 hr at room temperature. Unbound antibody was removed by washing as describe before and the color was developed using TMB solution (100 ml/well). After 5 min the reaction was terminated by the addition of 1M H₂SO4 (50 μl/well) and the optical densities were measured at 450 nm. The cutoff value was determined as the mean value of 10 healthy individual serum sample (for the protein depicted in FIG. 1) plus 2 standard deviation values.

Hybridization of Oligonucleotide Conjugates with Cy5-Oligonucleotide Probe

Arrays of cellulose binding regions (SEQ ID NO 4) non-conjugated and covalently bound to oligonuclotides attached to cellulose membranes were prepared and then washed for 30 min in room temperature with pre-hybridization solution (150 mM sodium citrate, 5× Denhardt's solution, pH8). The prehybridization solution was removed and hybridization solution (150 mM sodium citrate, 5× Denhardt's solution, pH 8) containing 5 μg/ml of 5′ end Cy5 oligonucleotide probe was applied on the array. Following 3 h hybridization at 37° C. under continuous mixing, the array was washed twice in 100 mM sodium citrate pH 8, 10 min each wash, following a brief rinse in 1×SSC.

### Example 1

### Cloning, Expression and Purification of a Cellulose Binding Recombinant Protein

A plasmid containing the gene encoding the cellulose binding region of Clostridium thermocellum cellulosome (SEQ ID NO 1) and its native N-terminus (SEQ ID NO 2) and C-terminus linkers, (SEQ ID NO 3) was used to transform E. Coli strain BL21 (DE3; Novagen, WI, USA). The transformed cells were grown on LB medium with appropriate antibiotics and IPTG (induction) for 12 to 18 hr at 16° C. without induction. Cell culture was centrifuged, resuspended in Tris buffer (50 mM, pH 7.2), sonicated and centrifuged again. Microcrystalline cellulose (Sigma Chemical Co., St. Louis, Mo., USA) was added to the clear supernatant, and the suspension was stirred for 1 h. After centrifugation, the pellet was washed once with phosphate-buffered saline (PBS) containing 1 M NaCl and twice with PBS. The recombinant protein was eluted from the cellulose matrix with 1% triethylamine, neutralized to pH 7 and stored at −20° C.

### Example 2

### Cloning, Expression and Purification of a Recombinant Fusion Protein

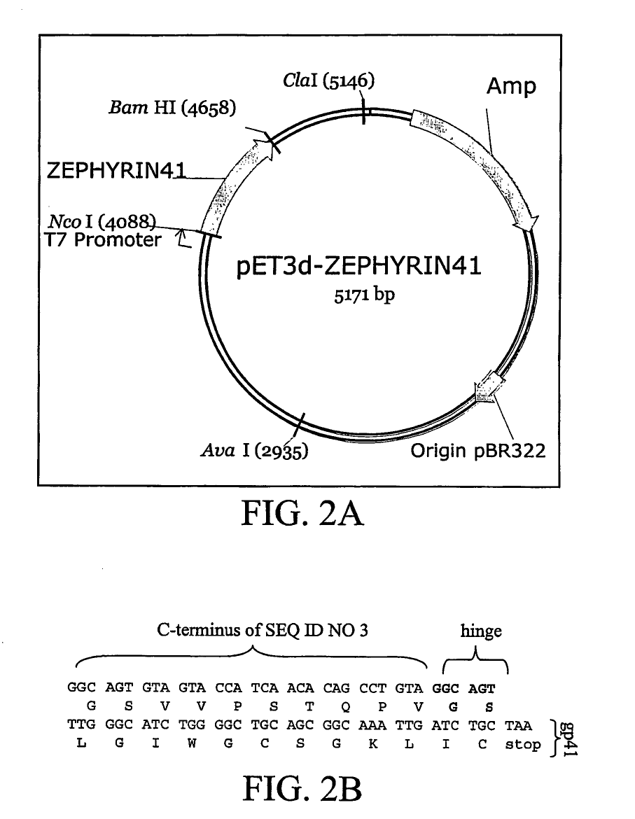

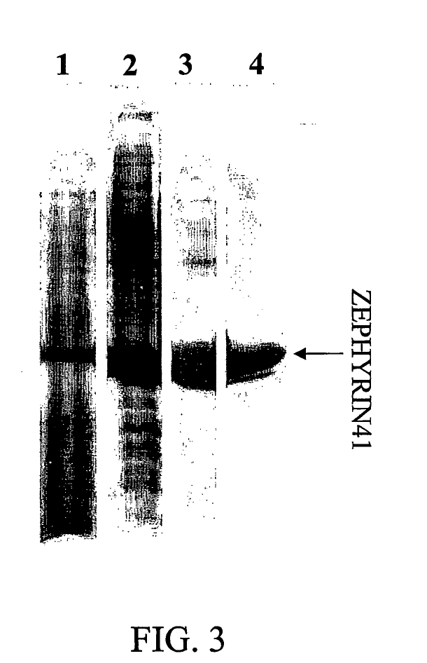

The C-linker of the cellulose binding recombinant protein (EXAMPLE 1; SEQ ID NO 3) was genetically mutated by point mutation as follows (FIG. 2): a) residues 7-8 were mutated to give flexible hinge linker (GS); b) residues 9-20 were mutated by the sequence LGIWGCSGKLIC (SEQ ID NO 7) which represent ZEPHYRIN41 antigenic epitope in HIV type 1 (Gnann J W, Science. 237:1346, 1987); and c) residues 21-34 were deleted. The resulting sequence was cloned into pET3d plasmid (Novagen WI, USA; FIG. 2A), and the resulting plasmid was used to transfect E. Coli strain BL21 (DE3). The transformed cells were grown on LB medium with the appropriate antibiotic. Upon IPTG induction, the recombinant fusion protein, also termed ZEPHYRIN41, was expressed as a soluble protein (FIG. 3, Lane 2).

The protein was purified from sonicated bacterial cells by two different schemes as described in the methods section (FIG. 3). Both purification schemes resulted in highly purified protein molecule (FIG. 3, lanes 2-4). The purification by affinity chromatography to cellulose matrix demonstrated that this protein molecule retained the original ability to bind specifically to its target matrix and to be eluted by conditions previously described in the literature. The molecular weight of the purified product was in agreement with the theoretical calculated value.

### Example 3

### Peptide Library Construction

E. Coli strains XL1 blue was used as a host cell during the library construction, strain BL21(DE3) and the T7 RNA polymerase expression vector pET3d were used for the expression of soluble fusion proteins comprising a cellulose binding region encoded by SEQ ID NO 1 and a peptide. The sequence encoding the peptide library was synthesized by applying random mutagenesis on the 8 nucleic acids located at the C-terminus of SEQ ID NO 3 which is the C-terminus linker of SEQ ID NO 1. To avoid premature termination an NNB (B=C/T/G) degeneracy was used in order to exclude all stop codons. Amplification was performed using:

(SEQ ID NO 8)

primer I-
GAGATATATC ATGAGCGCTA ATACACCGGT ATCAGGC

(SEQ ID NO 9)

primer II-
TAGCAGGGAT CCTIAVNNVN NVNNVNNVNN VNNVNNVNNA
CTGCCTACAG GCTGTGTTGA TGG

template—SEQ ID NO 1 joined, in frame, with SEQ ID NO 3

The amplified PCR product was digested with BamHI and BspHI and was ligated to BamHI/NcoI digested pET3d (Novagen, Inc., Madison, Wis.). Chemically competent XL1 blue cells were transformed with 20 μl aliquots (10 ng). Transformed cells were grown overnight under appropriate conditions and plasmids were purified. Purified plasmids were used to transform competent BL21 cells. After transformation cells were plated on LB agar plate containing ampicillin (100 μg/ml) and were grown overnight. The peptide library was amplified by PCR.

The peptide library was expressed in E. Coli BL21 strain. 96 single colonies were randomly selected and removed into sterile 96-well plates, using sterile tips, where each well contained 100 μl LB medium enriched with ampicillin (100 μg/ml). Cells were grown under continuous agitation at 37° C. until about OD=1 at 600 nm and were then induced with IPTG (final concentration of 1 mM) and expression was carried on for 18 hr under continuous agitation at 16° C. Aliquot of SDS/NaOH (0.4%/80 nM) solution was added into every well and cell lysis was taking place. Cell lysate was then attached to a matrix of cellulose as described above.

### Example 4

### Immunoassay Assessment of the Recombinant Proteins

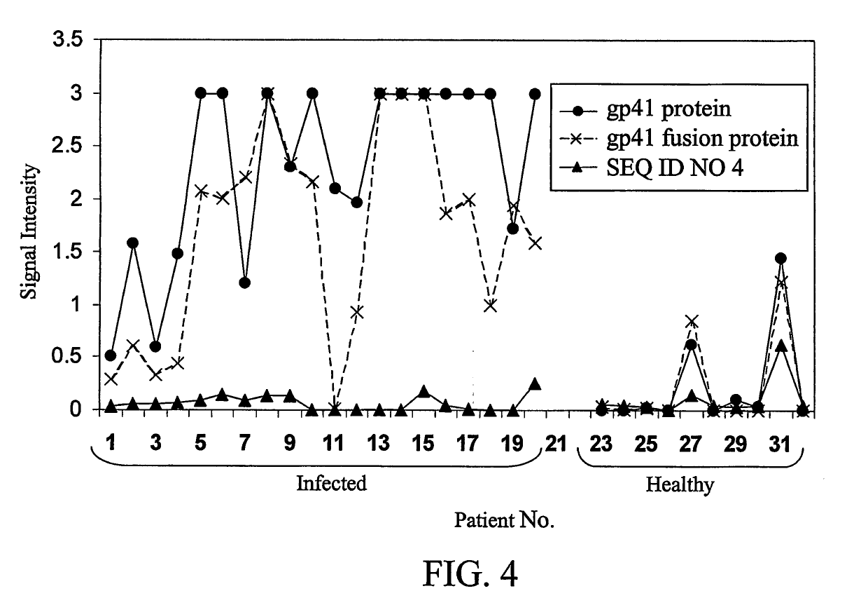

The sensitivity and specificity of purified ZEPHYRIN41 was tested in an immunoassay test with serum of HIV type 1 infected individuals (n=20; FIG. 4 patient Nos. 1-20) and with serum of healthy individuals (n=10; FIG. 4 patient Nos. 23-32). For this purpose micro-plate was coated, as described hereinabove, with the protein having the sequence SEQ ID NO 4, the recombinant mutated fusion protein (ZEPHYRIN41) and recombinant gp41 (see FIG. 3). Each sample of serum was diluted×10⁵.

Antibodies against ZEPHYRIN41 and recombinant gp41 were detected in 19 of 20 and in 20 of 20 HIV type 1 infected individuals respectively, and in 2 of 10 for both molecules in healthy individuals. In contrast, the cellulose binding protein (SEQ ID NO 4) was not reactive with serum sample from HIV type 1 infected individuals but a single false positive result (1 of 10) was detected in the samples from healthy individuals (FIG. 4; sample #31). A single false negative result (1 of 10) was observed for ZEPHYRIN41 in HIV type 1 infected individual serum. Two false positive results were detected for the recombinant gp41 and one for ZEPHYRIN41 in the healthy individual serum (FIG. 4; samples #27 and #31).

The results for the HIV type 1 infected individuals were consistent with those obtained from a commercial kit regularly used in hospitals for HIV type 1 detection These results clearly demonstrate the utility of ZEPHYRIN41 in term of sensitivity and specificity compared to the recombinant gp41 protein.

### Example 5

### Serodiagnosis of HIV Type 1 in Microarray Format—Model for Peptide Antibody Interaction

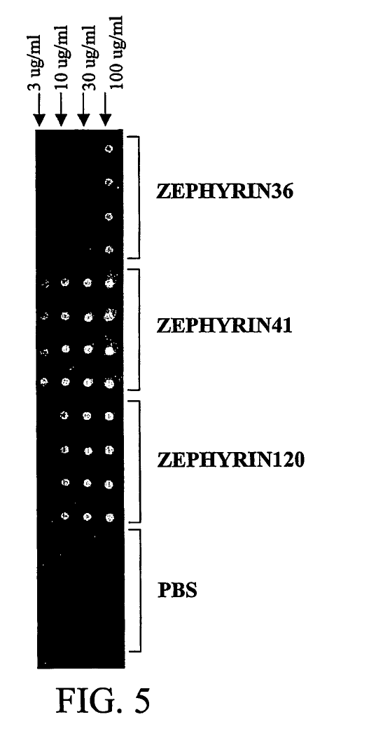

Antigen-peptide microarray for serodiagnosis of HIV type 1 and type 2 was prepared using the following recombinant proteins of the invention: ZEPHYRIN36 (for HIV type 2 only), ZEPHYRIN41 and ZEPHYRIN120 (for HIV type 1 and 2). The plasmid construction, expression and purification of ZEPHYRIN36 and ZEPHYRIN120 as was described above for ZEPHYRIN41 (EXAMPLE 2). The type of microarray described herein allows simultaneous detection of both antibodies directed against HIV type 1 and antibodies directed against HIV type 2. The recombinant proteins were fabricated in serial dilutions on cellulose-coated glass slide. In order to identify the specific human IgG that binds to the fabricated molecules, slides were incubated with Cy3 Goat anti-human antibody. Fluorescence measurement of the microarray incubated with HIV type 1 infected individual serum (FIGS. 5A-D) clearly indicated positive interaction with ZEPHYRIN41 and ZEPHYRIN120 molecules for a wide range of concentrations (3-100 μg/ml) and for ZEPHYRIN36 concentration of 100 μg/ml. The results for the HIV type 1 infected individuals were consistent with those obtained from a commercial kit regularly used in hospitals for HIV type 1 detection.

### Example 6

### Direct and Indirect Detection

Direct Detection with Cy5 Dye

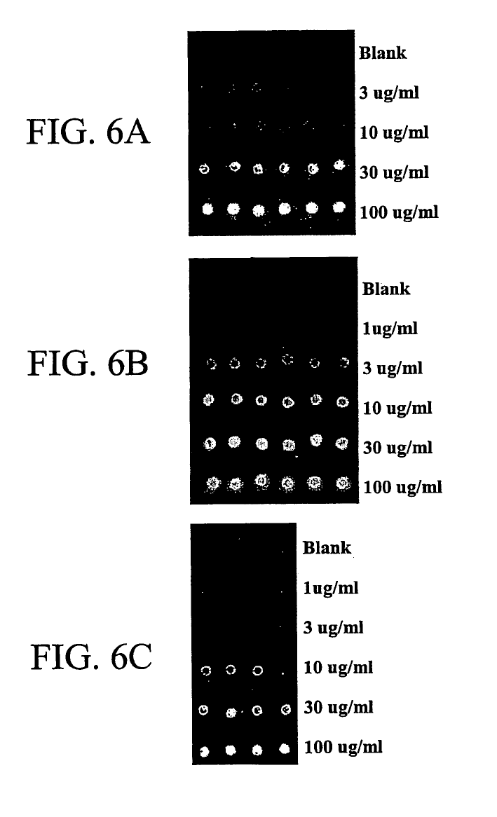

The recombinant protein comprising the cellulose binding region of SEQ ID NO 4 was tagged with Cy5 mono-reactive Dye Pack (Pharmacia biosciences; coupling through amine residue of lysine) prior to the fabrication as described hereinabove. The fabrication was followed by an extensive washing step and then the coated slide was blocked with 2% BSA (ICN, CA, USA) solution for 1 hr at room temperature. The washing and blocking steps were performed in complete darkness. Finally, the slide was analyzed using a microarray scanner as described hereinabove. FIG. 6A shows a representative response obtained by direct labeling of the recombinant protein containing the cellulose binding region. The image shows that direct labeling does not impair the adsorption of the labeled molecule to cellulase substrate. This method enabled the detection of low peptide concentration, 6 picogram protein per microarray spot.

Indirect Detection with Biotin-Streptavidin System

The recombinant protein comprising the cellulose binding region of SEQ ID NO 4 was labeled with Biotin employing conjugation through amine residue of lysine (Sigma Chemical Co., MO, USA), fabricated onto cellulose coated glass slide, and detected using Streptavidin Alexa fluor® 546 (Molecular Probes, OR, USA; FIG. 6B) as described hereinabove. Labeling of the recombinant protein molecule through amine residues did not impair its ability to bind cellulose. This method presented similar levels of detection as describe before (6 picogram protein per microarray spot).

Indirect Detection

The recombinant protein comprising the cellulose binding region of SEQ ID NO 4 was detected using Cy5 tagged rabbit antibody directed against the protein (FIG. 6C). The color intensity obtained from the Cy5 tag increased in line with the increase of recombinant protein concentration. This indirect quality control test enabled the detection of about 2 picogram of recombinant protein molecule per spot in the microarray. The fluorescence resulting from the non-biotinylated recombinant protein and from PBS in the second and third detection assays was negligible, indicating a very low degree of non-specificity.

### Example 7

### Fabrication of the Microarray with Non-Purified Probes/Molecules

Microarray fabrication of a protein or a peptide by the technologies known in the art requires purification of these molecules prior to the fabrication process. This prerequisite is definitely impractical for the fabrication of libraries containing thousands of proteins. The microarray of the present invention provide a practical and efficient alternative as shown hereinbelow.

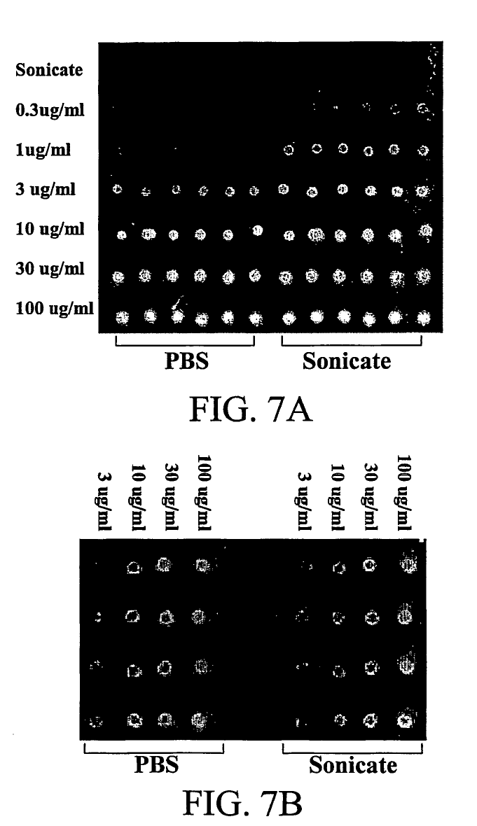

A microarray, fabricated on cellulose-coated glass slide, was constructed using a recombinant protein containing cellulose binding region of SEQ ID NO 4 diluted with E. Coli extract (FIG. 7A; Right panel) or PBS (FIG. 7A; Left panel). The fabrication, blocking, and analyses were performed as described hereinabove. Immobilization of the recombinant protein to cellulose coated glass slide was evaluated using the fluorescence signals generated when Cy5 rabbit antibody against the recombinant protein was introduced to the microarray (FIG. 7A). Relatively similar extent of binding to cellulose was observed for both the purified and the non-purified recombinant protein comprising the cellulose binding region having the sequence of SEQ ID NO 4. At low concentrations (0.3-3 μg/ml) binding of non-purified recombinant protein was higher than binding of the purified recombinant protein. The increased sensitivity which was demonstrated at very low protein concentrations may be interpreted as follows. The purified biotinylated-protein demonstrated low levels of non-specific binding to the solid support which thus became significant only at low concentrations of the protein. However, the E. Coli cell extract that was present in the non-purified biotinylated-protein samples blocked the solid support in a way that minimized the non-specific binding.

The results here indicate that binding of proteins comprising the cellulose binding region of SEQ ID NO 4 or the cellulose binding region of SEQ ID NO 4 flanked by at least one of the native linkers (i.e. SEQ ID NO 5 and/or SEQ ID NO 6) to cellulose, is not affected by the presence of unrelated proteins, DNA and other small molecules.

Efficient binding of non-purified recombinant protein was also shown in a microarray based on recombinant proteins comprising the cellulose binding region of SEQ ID NO 4 as well as in a microarray based on recombinant proteins comprising the cellulose binding region of SEQ ID NO 4 flanked by SEQ ID NO 6, labeled with His₆-tag (FIG. 7B). Binding was assessed using Cy5 mouse anti his₆-Tag antibody (Serotec Ltd., Oxford, UK). Again, the presence of crude E. Coli extract did not impair the extent of binding of the recombinant protein labeled with His₆-tag to cellulose (FIG. 7B).

Fluorescence of E. Coli extract alone, using both detection methods described in the present example (FIGS. 7A and 7B), gave no signal. This result emphasized the finding that binding of non-purified recombinant protein comprising the cellulose binding region of SEQ ID NO 4 as well as in a microarray based on recombinant proteins comprising the cellulose binding region of SEQ ID NO 4 flanked by SEQ ID NO 6, in E. Coli extract to cellulose, is highly specific and that the cellulose matrix remains inert to the nonspecific adsorption of various molecules present in the crude extract. The results presented in this example clearly demonstrate the superiority of the microarray of the present invention for construction of peptide and protein libraries and for high throughput applications, over the exiting technologies.

### Example 8

### Microarray Application for a Protein and a Small Ligand Interaction

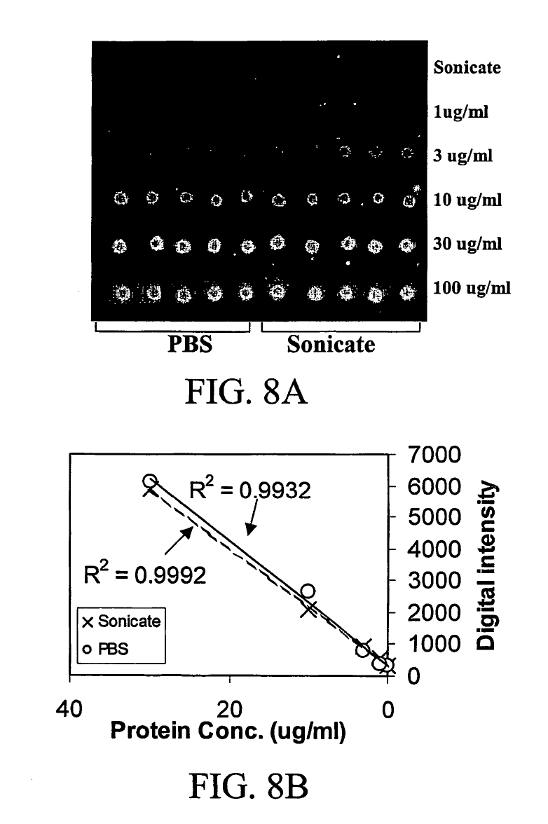

The interaction of a small ligand and a protein using the microarray of the present invention was tested. For this purpose biotinylated recombinant protein comprising the cellulose binding region of SEQ ID NO 4 was diluted with PBS to concentrations of 1, 3, 10, 30 and 100 μg/ml. These protein samples were fabricated in six replicates and the interaction with Streptavidin Alexa flour® 546 was measured (FIG. 8A). Linear regression analysis was applied on the photo-multiplier counts versus the concentration of the biotinylated recombinant protein fabricated on the cellulose coated glass slide (FIG. 8B). The correlation coefficient (R²) calculated from this analysis was close to unity. In most samples a coefficient of variation (CV) for each concentration was less than 15%.

### Example 9

### Microarray Dual Application: Detection and Interaction

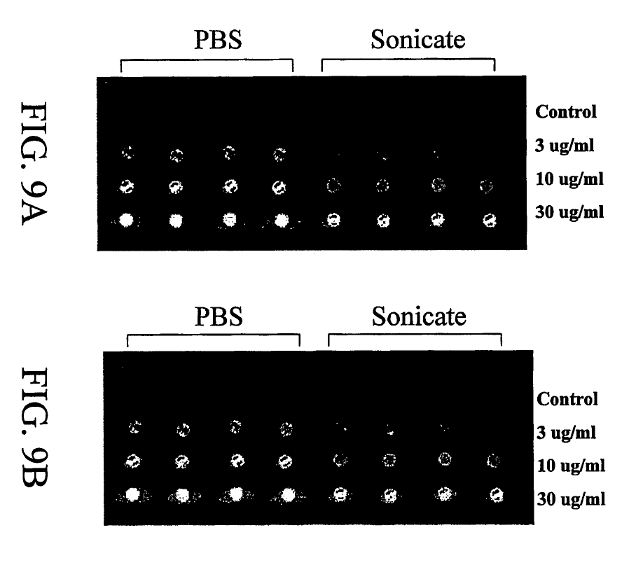

It was demonstrated that applying the microarray of the invention enables quantitative and qualitative detection by direct and indirect labeling methods (Example 5). It therefore became of an interest to determine the ability to detect a molecule and simultaneously to perform an immunoassay test by the same molecule using the microarray of the invention. For this purpose a microarray of ZEPHYRIN41 was fabricated onto a cellulose-coated glass slide. Samples of sera from HIV type 1 infected individuals were applied onto the microarray, following incubation with Cy3 goat anti-human IgG (FIG. 9A) and Cy5 rabbit directed against the protein of SEQ ID NO 4 (FIG. 9B). The simultaneous incubation step was followed by washing and scanning steps. This example established for the first time a new strategy for simultaneous measurement with no mutual interference of two different antibodies for two different epitopes in one molecule.

### Example 10

### Cell Adhesion Assay Using C-ZEPHYRIN-α4

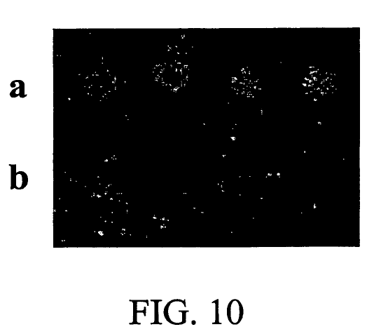

The interaction of cell surface receptor and small ligand such as peptide was used as a model system to demonstrate the compatibility of the microarray of the present invention to cell adhesion assay. The model of interaction chosen for this purpose was α4β1-CS1-interaction model. α4β1 is an integrin expressed by several types of adherent cells and can mediate cell matrix contact trough the known ligand fibronectin. The known binding region sites within this protein in the CS1 peptide. Accordingly, the C-linker (SEQ ID NO 6) of the protein of SEQ ID NO 4 was genetically mutated by point mutation to display the CS1 peptide (EILDVPST; SEQ ID NO 10). The resulting protein, named herein ZEPHYRIN-CS1 and the original protein were used to fabricate cellulose-coated glass slide. Jurkat cells expressing the α4β1 integrin which were labeled (by fluorescence) were then introduced with the microarray and allowed to interact with the ZEPHYRIN-CS1 for 1 hr at room temperature. The slides were washed extensively and the interaction was scanned by the microarray scanner (FIG. 10). As anticipated, only spots containing the ZEPHYRIN-CS1 were clearly visible, indicating that the labeled cells adsorbed to this molecule, by virtue of its selective affinity to the α4β1 integrin expressed on the cells. The binding was found to be proportional to the concentration of ZEPHYRIN-CS1. Negative control spots containing the original protein (SEQ ID NO 4) at the highest array concentration, yielded no detectable signal.

### Example 11

### Stability of the Microarrays under Extreme Conditions

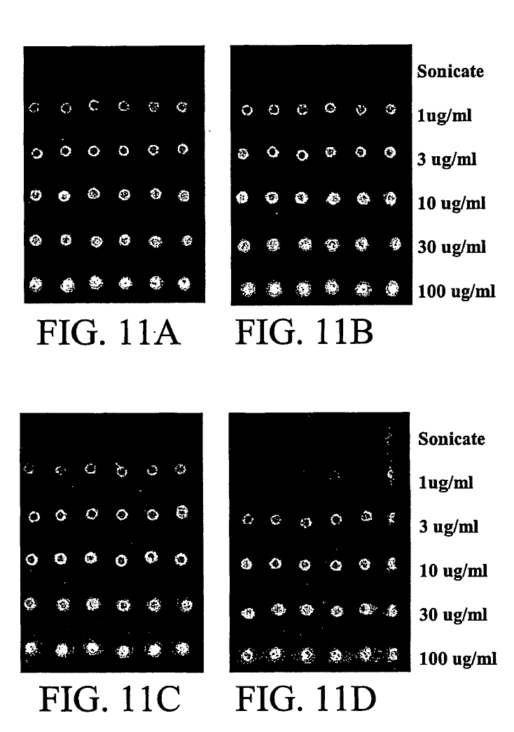

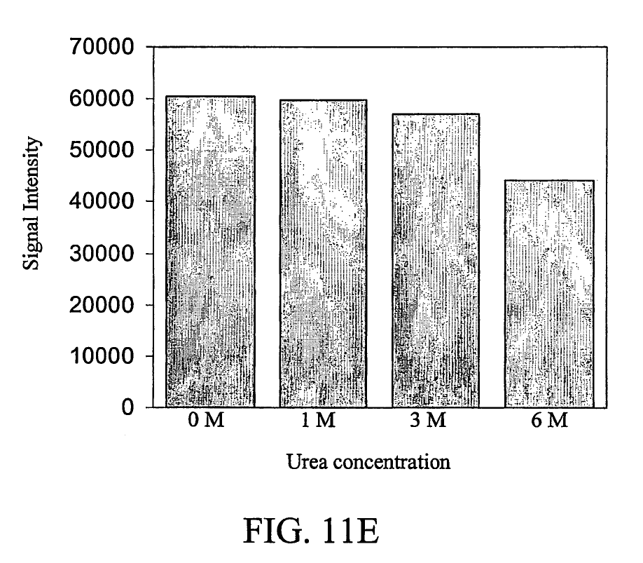

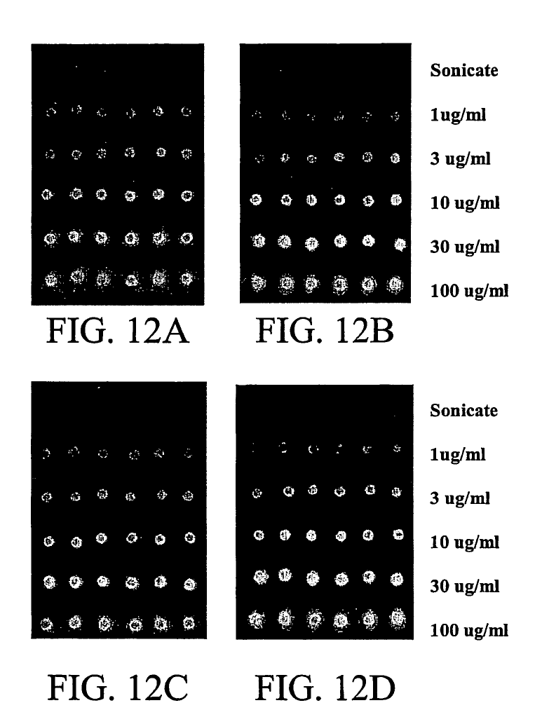

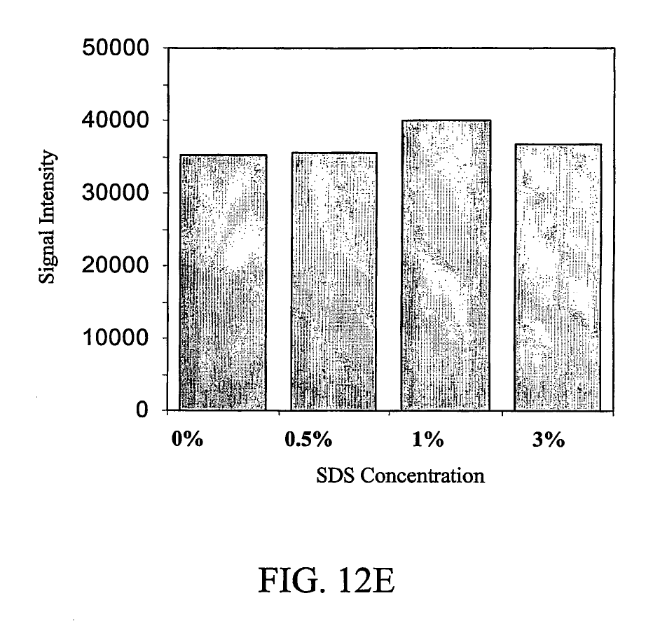

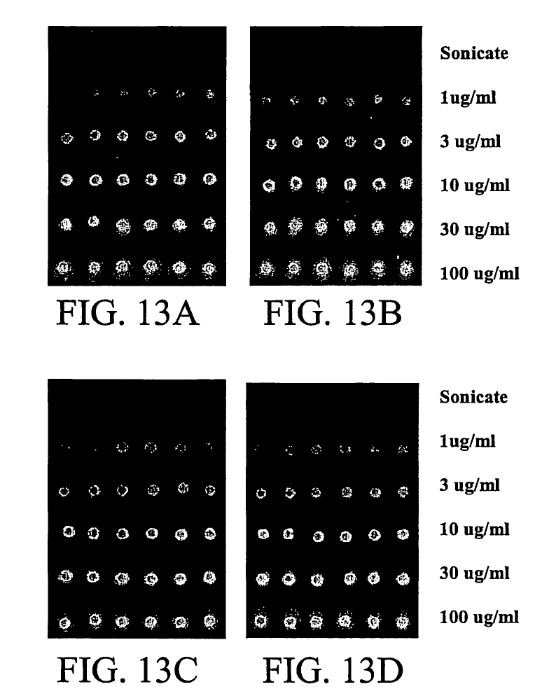

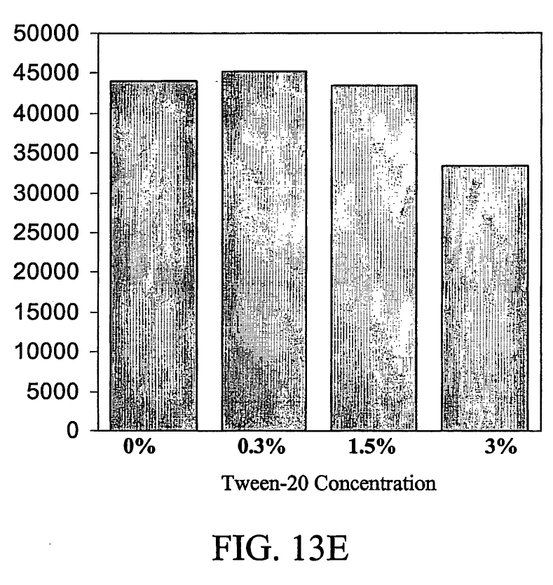

Ligand characterization may require solubilization, stabilization, purification and activation under harsh conditions using detergents such as SDS, Tween-20 and chaotropic agents such as urea. To demonstrate the capability of the microarrays of the present invention to perform under extreme conditions, proteins having the amino acid sequence of SEQ ID NO 4 chemically bound to biotin were attached to cellulose coated glass slides and were incubated for 1 hr at various concentrations of urea (FIG. 11), SDS (FIG. 12) and Tween-20 (FIG. 13). Slides were then incubated with streptavidin alexa fluor 546® and the residual amount of immobilized protein was measured as shown in FIG. 11E (urea), FIG. 12E (SDS) and FIG. 13E (Tween 20). Binding of the proteins to cellulose was not affected by any of the tested agents not even when extreme concentrations were used (e.g. 6 M urea, 3% SDS). The results demonstrate the high stability of the microarrays of the present invention.

### Example 12

### DNA Microarrays

A microarray of oligonucleotides conjugated to Cysteine 55 of proteins comprising SEQ ID NO 4 (the only cysteine in SEQ ID NO 4) was used. Coupling was carried through the free sulfhydryl group of Cysteine 55. The conjugates were prepared as follows: aliquots of 10 μl (2 mg/ml) of 24-mer 5′ end amino modified oligonucleotide were mixed with 2 μl of 1M NaHCO₃ pH8.1 and 8 μl (0.7 mg/ml) Sulfo-SMCC. The mixture allowed to react at room temperature for 1 hr. The activated-oligonucleotides were then mixed with 16 μl (6 mg/ml) of proteins having SEQ ID NO 4 and maleimide at the molar ratio of 4:1 activated-oligonucleotide to protein following 4 hr incubation at room temperature. Conjugates were stored at 4° C.

Conjugates and non-conjugated proteins (SEQ ID NO 4) were attached to cellulose coated glass slides. Following pre-hybridization step the cellulose-coated slides were incubated with complementary Cy5 oligonucleotide probe, washed and scanned.

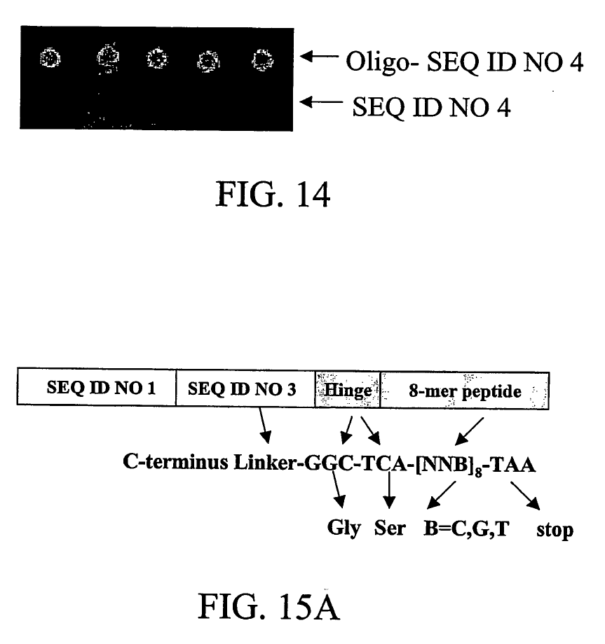

The results, as shown in FIG. 14, clearly indicate the specific binding of Cy5 oligonucleotide probe to its associated molecule, i.e. to proteins comprising a cellulose binding region (SEQ ID NO: 4) coupled through the free sulfhydryl group of cysteine 55 to an oligonucleotide. Non-conjugated proteins which contained only SEQ ID NO 4 did not produce fluorescence signals, demonstrating that the technology has both good signal-to-noise ratio and high specificity. The interaction of an array containing non-conjugated 24-mer amino modified oligonucleotides with Cy5 probe was non-detectable. This example establishes a new strategy for DNA microarrays based on cellulose binding proteins with improved presentation and orientation of the oligonucleotides suitable for DNA-DNA interaction.

### Example 13

### Microarrays of Peptide Library

Peptide microarrays enable precise characterization of molecular recognition events at the amino acid level. In addition, information obtained from peptide-microarrays can be used for measuring enzymatic activity such as protease activity, kinases and isomerases, and may be also used for cell adhesion assay. Peptides library are collections of very large numbers of random recombinant peptides, in which almost all possible combinations of the amino-acids used are represented. Numerous techniques for peptide library synthesis are known. Yet, the ability to fabricate pre-synthesized or in-situ synthesized peptides on the surface of glass-slide is limited to hundreds of peptides per slide. The following example demonstrates recombinant display of random peptide library on cellulose-coated glass slide using the microarrays of the invention.

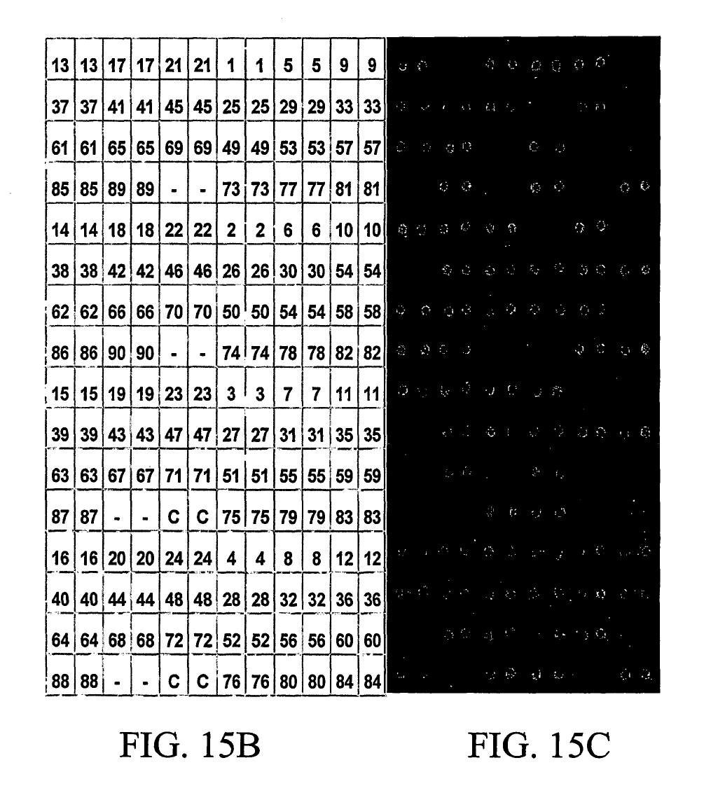

A combinatorial library was constructed at the C-terminus linker (SEQ ID NO 3) of SEQ ID NO 4. The randomization procedure resulted in a display of random peptides of 8 amino-acids (FIG. 15A). Plasmids containing the DNA sequence encoding SEQ ID NO 1 and the mutated SEQ ID NO 3 in the same reading frame were used to transform purified E. Coli strain BL21. The transformed cells were plated on LB agar plate for overnight incubation, single colonies (96 colonies) were then randomly selected and transferred into 96-well plate containing 100 μl of medium. Cells were induced using IPTG and following overnight incubation cells were lyzed using SDS/NaOH solution and cell lysate was fabricated in duplicates (FIGS. 15B-C). The peptide library was detected using Cy5 labeled rabbit anti SEQ ID NO 1 (FIG. 15C). Most colonies were readily detected by the labeled antibody. The signal intensities was relatively uniform indicating uniform expression level for the positive colonies that were grown under identical conditions in the 96-well plate. In addition, the harsh condition of solubilization of the cells in the 96-well plate did not affected the binding of the proteins comprising the peptide library to the cellulose matrix. Sequence analysis of the clones showed that no biases were found using the library construction.

### Example 14

### Construction of Cellulose Binding Regions Containing Exogenous Peptides

The principle of modifying a cellulose binding region such that it further contains exogenous peptides is exemplified herein using a protein encoded by SEQ ID NO 1 modified at loop 5/6.

DNA primers with homology to the C terminus of SEQ ID NO 1 and with partial homology to the region of loop 5/6 within that sequence were synthesized:

(SEQ ID NO 11)

terminal primer III:
ccgcaccatg gcaaatacac cggtattca

(SEQ ID NO 12)

terminal primer IV:
tcgcggatcc ttatactaca ctgccaccgg g

(SEQ ID NO 13)

loop 5/6 primer V:
gcccgttgaa accgtgccgg tcacttttac aaatgttcct tt

(SEQ ID NO 14)

loop 5/6 primer VI:
gtgaccggca cggtttcaac gggcgacacc taccttgaaa ta

The primers for remodeling the loop 5/6 were designed to contain the following amino acid: VTGTVSTG (SEQ ID NO 15). Amplification of the remodeled region DNA via PCR was performed for 25 cycles with ExTaq polymerase (TAKARA BIO Inc., JAPAN). The PCR products were purified from agarose gel and used for a second amplification round with the N and C terminal primers. The PCR products of the second run were digested by NCO I/BamHI and ligated.

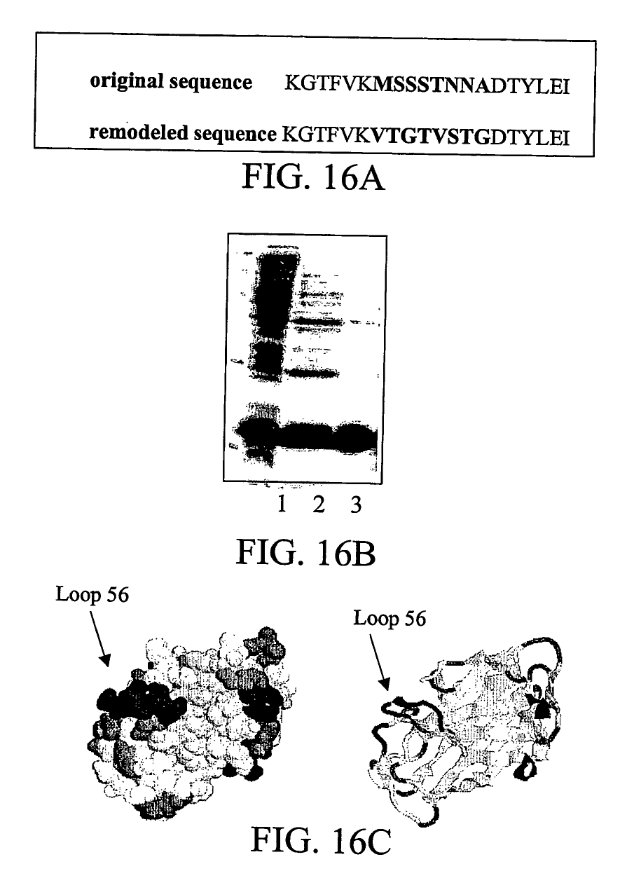

Loop 5/6 (LP5/6) within SEQ ID NO 4, is located between β-strand 5 and β-strand 6. In this example a random peptide of 8 amino-acids was introduced into this loop using molecular biology techniques. The original loop sequence MSSSTNNA (SEQ ID NO 16) was mutated to give the following sequence: VTGTVSTG (SEQ ID NO 15), which is completely incomparable with the original sequence. The overall structural organization of the original and the LP5/6-modified fragment is illustrated in FIG. 16A. The modified sequence was cloned into pET3d plasmid and the resulting plasmid was used for protein expression. Upon IPTG induction the mutated protein was expressed in a soluble form (FIG. 16B, lane 1). The stability of the LP5/6-mutated protein at 60° C. (FIG. 16B, lane 2) and its binding efficiency to cellulose (FIG. 16B, lane 3) were investigated. The LP5/6-mutated proteins were biotinylated and attached to cellulose-coated glass slides. Binding to cellulose and microarray compatibility were compared to those of the non-modified protein. The binding of the LP5/6-modified protein to cellulose coated glass slide was similar to that of the original non-modified protein. LP5/6-modified protein also maintained selective binding activity for cellulose and was easily detected using an antibody directed against SEQ ID NO 4.

Additional sites within the cellulose binding region derived from Clostridium Cellulovorans which may be modified while maintaining the high affinity of the modified region to cellulase substrates are:
1) loop between β-strand 7 and 8
2) β-strand 8 and β-strand 9
3) loop between β-strand 3 and 4
4) the residues that form second conserved site, located in a shallow groove on the other side of the cellulose-biding surface. The residues forming this shallow groove are characterized by sugar-binding sites of lectins and glycosyl hydrolases, as well as by antigen-binding sites of antibodies and MHC molecules.

The concept of a cellulose binding region containing an exogenous peptide has a great potential for measurements of protein-peptide interaction. Small peptides that are used for protein recognition tend to have limited affinities and specificities preliminary due to the absence of a 3D-structure. Thus, presentation of peptides within a defined structure may increase recognition and binding with high affinity of such peptides.

### Example 15

### Microarray Application for a Protein—Protein Interaction

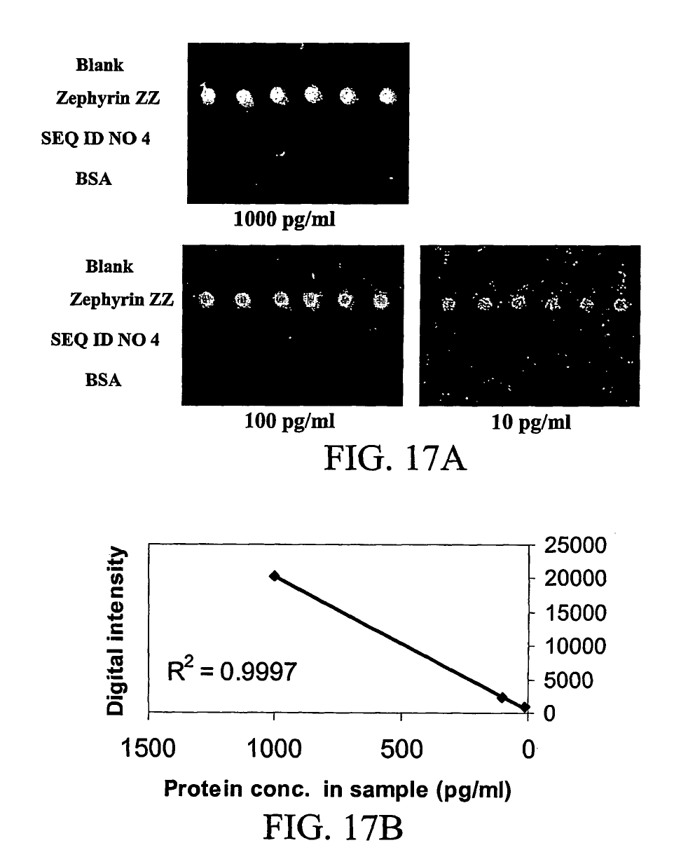

This example demonstrates the versatility of the microarray of the present invention towards the field of protein-protein interaction. A microarray of the protein of SEQ ID NO 4 fused to ZZ domain (two identical domains of protein A molecule) also termed hereinafter ZEPHYRIN-ZZ was fabricated onto cellulose coated glass slides. The spots consisted of 30 μg/ml of ZEPHYRIN-ZZ were allowed to react with 10, 100 and 1000 pg/ml of Cy5-rabbit IgG. FIG. 17A shows a set of 3 microarray fluorescence images, which were taken after 2-hr incubation of the cellulose-coated slides with the Cy5-rabbit IgG. The fluorescence signals increased with increasing concentrations of the antibody, whereas the intensities observed for negative control samples (SEQ ID NO 4) remained negligible. FIG. 17B shows the dose-response curves for the three concentrations (10, 100, 1000 pg/ml). Under the applied conditions, the dose-response curve was linear with a correlation coefficient of 0.999.

### Example 16

### Microarrays of Binary Ligand Display

A microarray of binary ligand display comprises: peptide library (as described in Example 13) and heparin molecules.

The construction of the microarray is based on three steps:
1. Amidation of heparin.
2. Production of maleimide activated heparin.
3. Coupling the heparin to the peptide library.

In the first step, amine group is introduced into the reducing end of the heparin molecule. For this purpose, heparin in phosphate buffer is mixed with adipic dihydrazide and allowed to interact for 5 days at 55° C. The amine-heparin is extensively dialyzed against PBS.

In the second step, the terminal amine-heparin is allowed to interact for 2 hr at room temperature with sulfosuccinnimidyl 4-(N-maleimidomethyl)-cyclohexane-1carboxylate). This water-soluble cross-linker is reacted with the amine group of the heparin molecule to give maleimide activated heparin molecule.

In the third step, the maleimide activated heparin is used for coupling the heparin to a peptide library or alternatively to protein fused to SEQ ID NO 4. The peptide library is a library of proteins comprising cellulose binding region and further containing a peptide inserted within the region or the peptide library is a library of fusion proteins wherein a first protein comprises a cellulose binding region and fused to a peptide. Prior to the interaction between the activated heparin and the peptide library, the library is attached onto a cellulose coated solid support. The coupling is carried through the free sulfhydryl group (Cysteine 55).

The screening step include labeling FGF with Cy dye such as Cy3 or Cy5. The labeled FGF is then added into BSA/PBS buffer and allowed to react with the fabricated microarray. After 1 hr incubation the cellulose-coated slide is washed extensively and signal is detected.

### Example 17

### Microarray Stability in the Presence of a Lysis Buffer

In order to obtain maximal efficiency of the peptide-library microarray the expression system of the linear peptide-library step has to be compatible with the high throughput requirement This requirement was examined using the microarrays of the present invention and a commercial lysis buffer consisting of NaOH and SDS, both of which are considered destructive agents but are also highly efficient and amenable for high throughput applications.

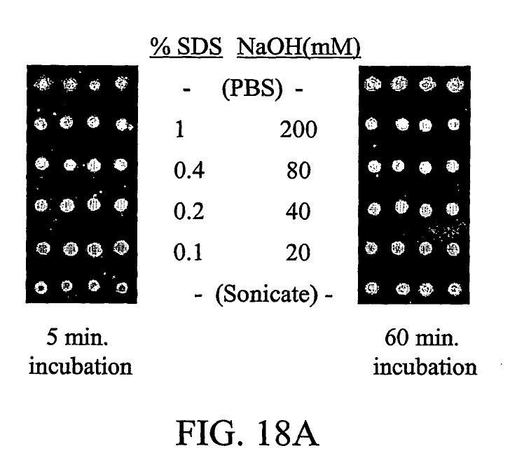

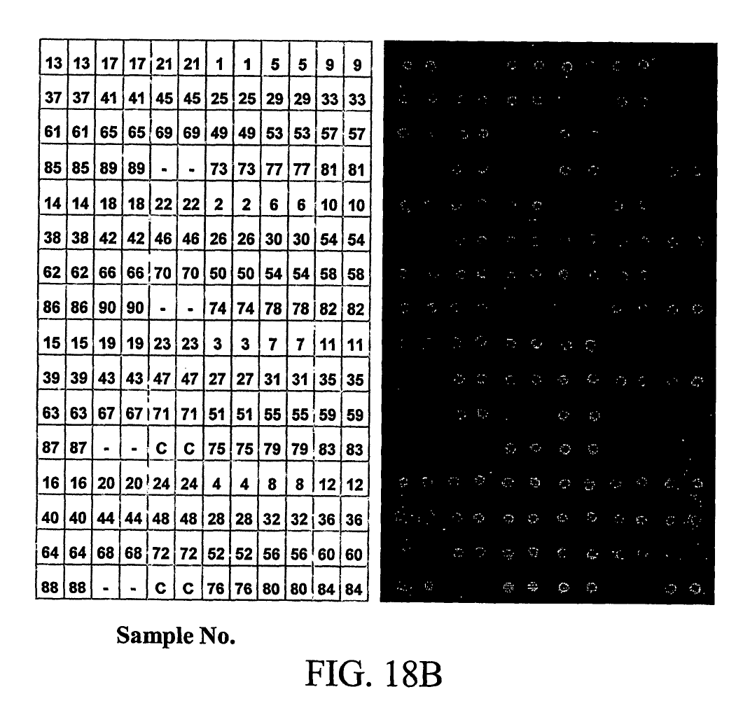

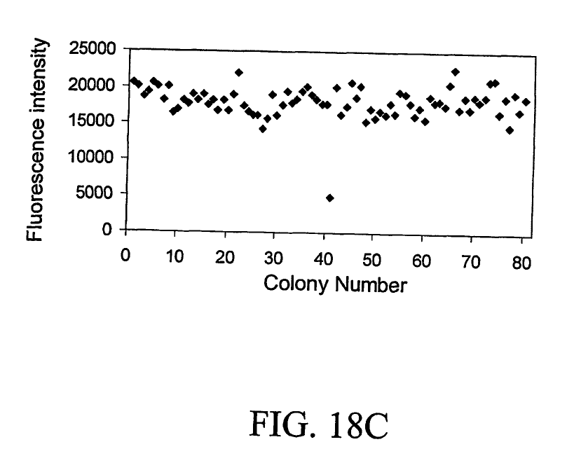

SEQ ID NO 4 biotinylated at the C-terminus was dissolved and incubated in lysis buffer or diluted lysis buffer for 5 or 60 min, following deposition on a cellulose-coated slide. The immobilized samples were detected by fluorescent streptavidin (FIG. 18A). The fluorescence signals generated from binding the biotinylated molecules to the cellulose membrane were not affected by the incubation time in the lysis buffer or by the concentration of the lysis buffer. Nevertheless, complete lysis of cells was clearly evident for all concentrations of lysis buffer.

Next, E. Coli were cultured in sterile 96-well plates containing 100-μl/well of growth medium and inoculated with 5-μl suspensions of single colonies expressing SEQ ID NO 4-linked to linear peptides of unknown sequence. The growth rate in the wells was monitored optically, using an ELISA reader equipped with a 600 nm filter. Upon reaching an OD 1, IPTG was added, and the induction was allowed continue for an additional 18 hr at 16° C. The cells were then lyzed by addition of 10-μl lysis buffer. Samples from each well (n=80) were fabricated in duplicates on cellulose-coated slides and visualized by Cy5-rabbit-anti-SEQ ID NO 4 (FIG. 18B). The expression level and the reproducibility were then estimated (FIG. 18C). Most of the wells generated positive and measurable signals after labeling the slide with the fluorescent antibody. High reproducibility scores were obtained (FIG. 18C) with an average signal intensity of 18,000 a.u. (arbitrary units) and standard deviation of 2,222 a.u.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

1. An array comprising a plurality of chimeric proteins, each protein comprising a cellulose binding region, wherein the cellulose binding region is devoid of cellulase activity; the proteins being immobilized onto discrete locations on a solid surface comprising a polymer capable of binding the cellulose binding region; with each location having at least one the protein displayed within the location.

2. The array according to claim 1, wherein the polymer is a homopolymer or a heteropolymer comprising polysaccharides selected from the group consisting of cellulose acetate, microcrystalline cellulose, lignin, starch, and xylane.

3. The array according to claim 1 wherein the cellulose binding region is derived from a protein other than a member of a polysaccharidase complex.

4. The array according to claim 3, wherein the cellulose binding region is derived from Clostridium Thermocellum.

5. The array according to claim 1, wherein the cellulose binding region comprises the amino acid sequence of SEQ ID NO 4.

6. The array according to claim 5, wherein the cellulose binding region further comprises at least one additional amino acid sequence of SEQ ID NO 5 or SEQ ID NO 6.

7. The array according to claim 1, wherein the cellulose binding region is encoded by a polynucleotide sequence comprising SEQ ID NO 1.

8. The array according to claim 7, wherein the polynucleotide sequence further comprises at least one additional polynucleotide sequence of SEQ ID NO 2 or SEQ ID NO 3.

9. The array according to claim 1, wherein the cellulose binding region further contains at least one exogenous peptide introduced therein, wherein the protein comprising the cellulose binding region is immobilized onto the solid surface such that the at least one exogenous peptide is exposed to the external environment in an accessible orientation.

10. The array according to claim 9, wherein the at least one exogenous peptide comprises a sequence of 5 to 50 amino acids.

11. The array according to claim 1, each protein further comprising at least one biologically active moiety fused thereto to form a fusion protein that is immobilized onto the solid surface such that each biologically active moiety is exposed to an external environment in an accessible orientation.

12. The array according to claim 11, wherein each protein further comprises at least two biologically active moieties.

13. The array according to claim 12, wherein the biologically active moiety is an enzyme, peptide, polypeptide, antibody, antigen, antigenic epitope, polynucleotide, hormone, carbohydrate, lipid, phospholipid, or biotinylated probe.

14. The array according to claim 12, wherein the at least two biologically active moieties are the same or different from each other.

15. The array according to claim 1, wherein the array is spatially addressable.

16. The array according to claim 1, wherein the array is one- or two-dimensional.

17. The array according to claim 1, consisting essentially of a plurality of genetically modified proteins each comprising a cellulose binding region and cellulose.

18. The array according to claim 1, wherein each protein comprises a detectable label.

19. The array according to claim 18, wherein the amount of the detectable label is proportional to the amount of protein.

20. A method for generating an array comprising a plurality of chimeric proteins comprising a cellulose binding region devoid of cellulase activity wherein the cellulose binding region further contains at least one exogenous peptide introduced therein, comprising the following steps:
(a) providing a polynucleotide encoding a cellulose binding region;
(b) introducing at least one oligonucleotide sequence encoding at least one exogenous peptide into the polynucleotide of (a) at a predetermined location;
(c) expressing the modified polynucleotide of (b) to obtain a protein comprising a cellulose binding region wherein the cellulose binding region further contains at least one exogenous peptide introduced therein;
(d) determining the binding efficiency of the protein of (c) to a cellulose polymer;
(e) attaching a plurality of proteins of (d) onto discrete locations on a solid support comprising a cellulose polymer to form an array of immobilized proteins; and
(f) determining the exposure of the at least one exogenous peptide contained within the proteins of (d) to the external environment at an accessible orientation.

21. The method according to claim 20 wherein the cellulose binding region is derived from a protein other than a member of a polysaccharidase complex.

22. The method according to claim 21, wherein the cellulose binding region is derived from Clostridium Thermocellum.

23. The method according to claim 20, wherein the cellulose binding region comprises the amino acid sequence of SEQ ID NO 4.

24. The method according to claim 23, wherein the cellulose binding region further comprises at least one additional amino acid sequence of SEQ ID NO 5 or SEQ ID NO 6.

25. The method according to claim 20, wherein the cellulose binding region is encoded by a polynucleotide sequence comprising SEQ ID NO 1.

26. The method according to claim 25, wherein the polynucleotide sequence further comprises at least one additional polynucleotide sequence of SEQ ID NO 2 or SEQ ID NO 3.

27. The method according to claim 20, wherein the at least one exogenous peptide comprises a sequence of 5 to 50 amino acids.

28. The method according to claim 20, wherein the array is spatially addressable.

29. The method according to claim 21, wherein each protein comprises a detectable label.

30. The method according to claim 29, wherein the amount of the detectable label is proportional to the amount of protein.

31. A method for the production of an array comprising a plurality of chimeric proteins comprising a cellulose binding region, wherein each chimeric protein comprises at least one biologically active moiety while maintaining the capacity to bind a cellulose polymer, comprising the steps of:
(a) providing a polynucleotide comprising a nucleic acid encoding a cellulose binding region devoid of cellulase activity fused to at least one biologically active moiety;
(b) expressing the polynucleotide of (b) to obtain a fusion protein;
(c) determining the binding efficiency of the fusion protein of (b) to a cellulose polymer;
(d) attaching a plurality of fusion proteins of (c) onto discrete locations over a solid support comprising a cellulose polymer to form an array of immobilized fusion proteins; and
(e) determining the exposure of the at least one biological active moiety to the external environment at an accessible orientation.

32. A method for the production of an array comprising a plurality of chimeric proteins comprising a cellulose binding region capable of binding cellulose polymers with high affinity, wherein each chimeric protein comprises at least one additional biologically active moiety covalently linked to the cellulose binding region while maintaining the capacity to bind a cellulose polymer with high affinity and wherein the at least one biologically active moiety is exposed to the external environment in an accessible orientation while the protein is attached to a solid support, comprising the steps of:
(a) providing a polynucleotide comprising a nucleic acid encoding a cellulose binding region devoid of cellulase activity;
(b) expressing the polynucleotide of (a) to obtain a protein comprising a cellulose binding region devoid of cellulase activity;
(c) covalently linking the protein of (b) to at least one biologically active moiety to obtain a chimeric protein;
(d) determining the binding efficiency of the chimeric protein of (c) to a cellulose polymer;
(e) attaching a plurality of chimeric proteins of (d) onto discrete locations over a solid support comprising a cellulose polymer to form an array of immobilized chimeric proteins; and
(f) determining the exposure of the at least one biological active moiety to the external environment at an accessible orientation.

33. The method according to claim 32, wherein the at least one biologically active moiety is obtained by modifying a C-terminus or an N-terminus sequence of the cellulose binding region, wherein the terminus has essentially no cellulose binding activity.

34. The method according to claim 32 wherein the cellulose binding region is derived from a protein other than a member of a polysaccharidase complex.

35. The method according to claim 34, wherein the cellulose binding region is derived from Clostridium Thermocellum.

36. The method according to claim 32, wherein the cellulose binding region comprises the amino acid sequence of SEQ ID NO 4.

37. The method according to claim 36, wherein the cellulose binding region further comprises at least one additional amino acid sequence selected of SEQ ID NO 5 or SEQ ID NO 6.

38. The method according to claim 32, wherein the cellulose binding region is encoded by a polynucleotide sequence comprising SEQ ID NO 1.

39. The method according to claim 38, wherein the polynucleotide sequence further comprises at least one additional polynucleotide sequence of SEQ ID NO 2 or SEQ ID NO 3.

40. The method according to claim 32, wherein each chimeric protein comprises at least two biologically active moieties.

41. The method according to claim 40, wherein the at least two biologically active moieties are the same or different from each other.

42. The method according to claim 32, wherein the array is spatially addressable.

43. The method according to claim 32, wherein each protein comprising a detectable label.

44. The method according to claim 43, wherein the amount of the detectable label is proportional to the amount of protein.

45. The method according to claim 32, wherein the biologically active moiety is selected from the group consisting of enzymes, peptides, polypeptides, antibodies, antigens, antigenic epitopes, polynucleotides, hormones, carbohydrates, lipids, phospholipids, and biotinylated probes.

46. A method for screening a plurality of biologically active moieties for interactions with one or more components of a fluid sample comprising the steps of:
providing a plurality of chimeric proteins immobilized at discrete locations over a solid surface, wherein the surface comprises a cellulose polymer and with the chimeric proteins comprising a cellulose binding region which is devoid of cellulase activity and at least one exogenous peptide introduced therein or at least one biologically active moiety fused or covalently linked thereto thus forming an addressable array of the chimeric proteins at discrete locations such that the at least one exogenous peptide or biologically active moiety is exposed in an accessible orientation;
contacting the array with a sample to be analyzed;
detecting binding interactions between the array and the sample to be analyzed; and
determining the loci of interaction.

47. The method according to claim 46, wherein the cellulose binding region of each chimeric protein contains at least one exogenous peptide introduced therein to form an addressable array of locations such that each exogenous peptide is exposed in an accessible orientation to a fluid sample in an external environment.

48. The method according to claim 46, wherein the cellulose binding region is derived from a protein other than a member of a polysaccharidase complex.

49. The method according to claim 48, wherein the cellulose binding region is derived from Clostridium Thermocellum.

50. The method according to claim 46, wherein the cellulose binding region comprises the amino acid sequence SEQ ID NO 4.

51. The method according to claim 50, wherein the cellulose binding region further comprises at least one additional amino acid sequence selected from the group consisting of SEQ ID NO 5 and SEQ ID NO 6.

52. The method according to claim 46, wherein the cellulose binding region is encoded by a polynucleotide sequence comprising SEQ ID NO 1.

53. The method according to claim 52, wherein the polynucleotide sequence further comprises at least one additional polynucleotide sequence selected from the group consisting of SEQ ID NO 2 and SEQ ID NO 3.

54. The method according to claim 47, wherein the at least one exogenous peptide comprises a sequence of 5 to 50 amino acids.

55. The method according to claim 46, wherein the array is spatially addressable.

56. The method according to claim 46, wherein each protein comprises a detectable label.

57. The method according to claim 56, wherein the amount of the detectable label is proportional to the amount of protein.

58. The method according to claim 46, wherein the cellulose binding region of each chimeric protein includes at least one biologically active moiety fused or covalently linked thereto, and which further comprises forming an addressable array of the chimeric proteins at discrete locations, each having at least one biologically active moiety displayed at each the location such that the at least one biologically active moiety is exposable in an accessible orientation to a fluid sample in an external environment.

59. The method according to claim 58, wherein the at least one biologically active moiety is obtained by modifying a C-terminus or an N-terminus sequence of the cellulose binding region, wherein the terminus has essentially no cellulose binding activity.

60. The method according to claim 58, wherein each protein comprises at least two biologically active moieties.

61. The method according to claim 60, wherein the at least two biologically active moieties are the same or different from each other.

62. The method according to claim 58, wherein the biologically active moiety is selected from the group consisting of enzymes, peptides, polypeptides, antibodies, antigens, antigenic epitopes, polynucleotides, hormones, carbohydrates, lipids, phospholipids, and biotinylated probes.
